(19) United States
(12) Patent Application Publication
(10) Pub. No.: US 2016/0045487 A1
(43) Pub. Date: Feb. 18, 2016

(54) COMPOSITIONS AND METHODS FOR TREATING NEUROPATHY

(71) Applicant: CHILDEREN'S MEDICAL CENTER CORPORATION, Boston, MA (US)

(72) Inventor: Gabriel Corfas, Brookline, MA (US)

(21) Appl. No.: 14/780,322

(22) PCT Filed: Mar. 26, 2014
(86) PCT No.: PCT/US2014/031920

§ 371 Date: Sep. 25, 2015

Related U.S. Application Data
(60) Provisional application No. 61/805,838, filed on Mar. 27, 2013.

(51) Int. Cl. A61K 31/4706; A61K 38/18

(52) U.S. Cl. CPC A61K 31/4706; A61K 38/185

(57) ABSTRACT

The invention features XIB4035 for the treatment of large fiber neuropathy, and combinations of XIB4035 and GDNF for the treatment of both large and small fiber neuropathies.

### RELATED APPLICATIONS

The present application claims priority to, and the benefit under 35 U.S.C. §119(e) of U.S. provisional patent application No. 61/805,838, entitled “Compounds and Methods for Treating Large Fiber Neuropathy,” filed Mar. 27, 2013. The entire content of the aforementioned patent application is incorporated herein by this reference.

### STATEMENT OF RIGHTS TO INVENTIONS MADE UNDER FEDERALLY SPONSORED RESEARCH

This work was supported by the following grants from the National Institutes of Health, Grant Nos: 5R01NS035884. The government has certain rights in the invention.

### BACKGROUND OF THE INVENTION

Peripheral neuropathy refers to disorders of the peripheral nervous system. Small fiber neuropathy (SFN) is a disorder of peripheral nerves commonly found in patients with diabetes mellitus, HIV infection, or those receiving chemotherapy. Large fiber neuropathy affects sensory neurons, motor neurons, or both. Large fiber neuropathies manifest with the loss of joint position and vibration sense and sensory ataxia, whereas small fiber neuropathy manifests with the impairment of pain, temperature and autonomic functions. The complexity of disease etiology has led to a scarcity of effective treatments for large and small fiber neuropathies.

### SUMMARY OF THE INVENTION

As described below, the present invention features the use of XIB4035 for the treatment of neuropathy (optionally, large fiber neuropathy), and compositions comprising combinations of XIB4035 and a GFRα ligand (e.g., GDNF, neuturin (NRTN), artemin (ARTN), neublastin, and/or persephin) and uses thereof for the treatment of neuropathy (e.g., diabetic small fiber neuropathy, injury-associated neuropathy, alcoholism-associated neuropathy, lupus-related neuropathy, HIV-related neuropathy, large fiber neuropathy, a neuropathy associated with chemotherapy, enteric neuropathy).

In one aspect, the invention provides a method for treating a neuropathy, involving administering an effective amount of a combination of XIB4035 and a GFRα ligand to a subject in need thereof.

In one embodiment, the method involves administering to the subject an effective amount of XIB4035 or a compound of Tables 1-3, to treat a neuropathy (e.g., a large fiber neuropathy).

In certain embodiments, XIB4035 enhances the activity of a GFRα ligand that is GDNF, neublastin, neuturin (NRTN), artemin (ARTN) or persephin.

In another embodiment, XIB4035 enhances the activity of ligand-induced GFRα/Ret signaling.

Optionally, the GFRα is GFRα1, GFRα2, GFRα3 or GFRα4.

In some embodiments, the subject is identified as having a neuropathy, optionally, a diabetic small fiber neuropathy, an injury-associated neuropathy, an alcoholism-associated neuropathy, a lupus-related neuropathy, an HIV-related neuropathy, a large fiber neuropathy, a neuropathy associated with chemotherapy or an enteric neuropathy.

In certain embodiments, an effective amount of XIB4035 is between about 0.5 and 3 μM.

In some embodiments, an effective amount results in a plasma concentration of 6.92-16.93 ng/ml at 6-12 hours after dosage. Optionally, the effective amount of XIB4035 is 1.5 μM.

In certain embodiments, the amount of XIB4035 is sufficient to relieve symptoms of neuropathy.

In one embodiment, XIB4035 is administered systemically.

In additional embodiments, XIB4035 is administered orally, intravenously, intramuscular, subdermally or intrathecally.

In another embodiment, XIB4035 is administered once per day.

In one embodiment, GDNF is administered, and optionally, is administered locally.

In an additional embodiment, the GDNF polypeptide is administered by injection into a ventricle of the brain, into cerebrospinal fluid, or is administered locally using an implanted pump or matrix.

In another embodiment, GDNF is administered using an expression vector having a polynucleotide that encodes GDNF.

In certain embodiments, the expression involves a promoter that directs expression in muscle or skin.

In another embodiment, the subject is identified as having a large fiber or other neuropathy by electrodiagnostic testing, sensory, motor nerve conduction, F response, H reflex, needle electromyography (EMG), and/or clinical indications.

Another aspect of the invention provides a composition for the treatment of neuropathy that includes an effective amount of XIB4035 and GDNF.

A further aspect of the invention provides a kit that contains an effective amount of XIB4035 and GDNF.

Another aspect of the invention provides a method for improving nerve conduction velocity (NCV) in a subject having a neuropathy by administering to the subject an amount of XIB4035 sufficient to improve nerve conduction velocity in the subject.

In one embodiment, nerve conduction velocity is improved in the sciatic nerve of the subject. Optionally, the subject has a diabetic neuropathy.

In certain embodiments, the nerve conduction velocity is at least 40 m/sec, at least 41 m/sec, at least 42 m/sec, at least 43 m/sec, at least 44 m/sec or at least 45 m/sec.

An additional aspect of the invention provides a method for increasing sub-epidermal neural plexus (SNP) density in a subject having or at risk of having a neuropathy by administering to the subject an amount of XIB4035 sufficient to increase SNP density in the subject.

A related aspect of the invention provides a method for maintaining sub-epidermal neural plexus (SNP) density in a subject having or at risk of having a neuropathy by administering to the subject an amount of XIB4035 sufficient to maintain SNP density in the subject.

In certain embodiments, SNP density is at least 40/mm, at least 41/mm, at least 42/mm, at least 43/mm, at least 44/mm or at least 45/mm.

Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “GFRα ligand” is meant a polypeptide or fragment thereof that specifically binds GFRα and induces GFRα/RET receptor signaling. GFRα/RET receptor signaling is measured by assaying Ret-induced gene expression, Ret-phosphorylation, measuring neurite extension, and/or measuring cell survival in cells at risk of apoptosis.

By “GDNF polypeptide” is meant a polypeptide having 85% or greater sequence identity to NCBI Reference No. P39905 or a fragment thereof. The sequence of an exemplary GDNF polypeptide is provided below:

>sp|P39905|GDNF_HUMAN Glial cell line-derived neurotrophic factor OS=Homo sapiens

### GN=GDNF PE=1 SV=1

MKLWDVVAVCLVLLHTASAFPLPAGKRPPEAPAEDRSLGRRRAPFALSSD
SNMPEDYPDQFDDVMDFIQATIKRLKRSPDKQMAVLPRRERNRQAAAANP
ENSRGKGRRGQRGKNRGCVLTAIHLNVTDLGLGYETKEELIFRYCSGSCD
AAETTYDKILKNLSRNRRLVSDKVGQACCRPIAFDDDLSFLDDNLVYHIL
RKHSAKRCGCI

By “GDNF polynucleotide” is meant a nucleic acid molecule that encodes a GDNF polypeptide.

By “agent” is meant a peptide, nucleic acid molecule, or small compound.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. Examples of diseases include large and small fiber neuropathy. In one embodiment, a neuropathy described herein is not a small fiber neuropathy.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1E show that the over-expression of GDNF in the skin rescues the small fiber neuropathy phenotypes of GFAP-DN-erbB4 mice. FIG. 1A is a graph, which quantitates results of a hot plate (54° C.) test showing that the loss of thermal nociception in GFAP-DN-erbB4 mice is prevented by GDNF over-expression in keratinocytes (K14-GDNF). Only responses of GFAP-DN-erbB4 differ from those observed in the other three genotypes (ANOVA Tukey post-hoc n≧3; * p<0.001; error bars=SEM). FIG. 1B includes electron micrographs of transverse sections from the sciatic nerve show that Remak bundle structure in K14-GDNF mice and GFAP-DN-erbB4::K14-GDNF mice is similar to wild type while bundles are lost in GFAP-DN-erbB4 mice (scale bar=4 μm). FIG. 1C is a graph showing a quantification of nerve terminals in footpad skin at P30. (ANOVA Bonferroni post-hoc N=3; wild type vs K14-GDNF p=0.037; GFAP-DN-erbB4 vs GFAP-DN-erbB4::K14-GDNF p=0.039; wild type vs GFAP-DN-erbB4 p=0.142; wild type vs GFAP-DN-erbB4::K14-GDNF p=0.819; K14-GDNF vs GFAP-DN-erbB4::K14-GDNF p=0.134; K14-GDNF vs GFAP-DNerbB4 p=0.001; error bars=SEM). FIG. 1D is a micrograph showing the number of PGP9.5-positive nerve terminals in footpads at P30 is increased by GDNF overexpression (K14-GDNF) and reduced in GFAP-DN-erbB4 mice compared to double transgenic mice. Skin innervation in K14-GDNF::GFAP-DN-erbB4 is not different than in wild types (scale bar=25 μm).

FIG. 1E provides micrographs showing GFAP-DN-erbB4 mice lose IENF skin innervation by P35. Representative images of PGP9.5-positive nerve terminals in footpads of wild type mice (P35) and GFAP-DN-erbB4 mutant mice (P21 and P35) show that GFAP-DN-erbB4 mice progressively lose intra-epidermal nerve fibers (IENFs) (scale bar=100 μm). Nuclei are stained with DAPI.

FIGS. 2A-2C show that prophylactic topical treatment with XIB4035 prevents the loss of thermal nociception and nerve degeneration in GFAP-DN-erbB4 mice and reduces neuropathic symptoms in STZ-induced diabetic mice. FIG. 2B is a graph. GFAP-DN-erbB4 mice were treated with control cream or cream containing XIB4035 (1.5 mM) for a period of 4 weeks (P21-P49). Thermal nociception was analyzed by using a 54° C. hot plate test. Wild type mice treated with either the control cream or XIB4035 had similar latencies throughout the duration of the treatment. GFAP-DN-erbB4 mice treated with control cream had progressively longer withdrawal latencies over the duration of the treatment while GFAP-DN-erbB4 mice treated with XIB4035 had withdrawal latencies similar to wild type mice. (ANOVA Tukey post-hoc N=6; * GFAP-DN-erbB4 v. GFAP-DN-erbB4+XIB4035; p<0.001; error bars=SEM). FIG. 2B includes electron micrographs of transverse sections from the sciatic nerve of GFAP-DN-erbB4 mice treated for 4 weeks (P49) show that Remak bundle structure is lost in mutants treated with control cream but preserved in GFAP-DN-erbB4 mice treated with XIB4035. Remak bundle structure in wild types is not changed by the treatment. FIG. 2C is a graph. Mice were exposed to a single i.p. injection of STZ to induce diabetes, and once hyperglycemic, were treated with either control or XIB4035-containing cream daily. Starting eight weeks after treatment initiation, mice were tested once a week for thermal nociceptive responses using the Hargreaves test. Thermal nociceptive loss in diabetic mice is reduced by XIB4035 treatment (student's t-test N≧10; * p<0.05, ** p<0.01, *** p<0.001, error bars=SEM).

FIGS. 3A-3C show that XIB4035 acts as a disease modifying treatment, reducing neuropathic symptoms when treatment is initiated after disease onset, and needs recurrent application to maintain sensory function. FIG. 3A is a graph showing that when XIB4035 treatment of GFAP-DN-erbB4 mice was initiated after disease onset (P28) mice showed significant improvement in thermal nociception (54° C. hot plate) one week later (P35), and this was maintained as long as the treatment continued (3 weeks) (ANOVA Tukey post-hoc N≧7; * p<0.05, *** p<0.001). FIG. 3B is a graph showing that neuropathic symptoms returned if mice were treated beginning at P28 as in A) but then treatment was interrupted at P35 (ANOVA Tukey post-hoc N≧7; *** p<0.001). (P28, P35, P42, and P49), p<0.01 (P56), error bars=SEM). FIG. 3C includes a series of micrographs showing that XIB4035 treatment of GFAP-DN-erbB4 mice after disease onset (P28) did not rescue IENF density in hind paw skin. Representative images of PGP 9.5-positive IENF staining at P35 in GFAP-DN-erbB4 hind paw skin show lack of IENF density recovery when XIB4035 treatment is initiated at P28.

FIG. 3D includes a series of micrographs showing that XIB4035 treatment of GFAP-DN-erbB4 mice after disease onset (P28) rescued IB4 positive terminals in lamina II of the spinal cord dorsal horn. Left: Representative images of spinal cord sections from wild type mice treated with control cream P28-P35 show the normal appearance of TrpV1+ (red) and IB4+ (green) nerve terminals. Middle: images of GFAP-DN-erbB4 mice treated in the same way show complete absence of IB4 staining. Right: images of tissues from GFAP-DN-erbB4 mice treated with XIB4035-containing cream show clear presence of IB4+ fibers. No obvious difference in TrpV1 labeling was observed between wild type and mutant animals regardless of treatment.

FIG. 3E is a graph showing that XIB4035 does not alter thermal nociceptive function in wild type mice. Thermal nociceptive responses in wild type mice treated with either control or XIB4035 (1.5 mM) containing cream were compared using the hotplate test (51° C.) beginning at four weeks of age (p28) and continued to six weeks of age (P42). XIB4035 treatment did not modify this behavior (Student's Ttest n≧10; P28 (p=0.0671), P35 (p=0.8343, P42 (p=0.8492), error bars=SEM).

FIG. 3F includes graphs showing that GDNF-family ligands induces activation of the tyrosine hydroxylase promoter luciferase reporter in a dose dependent fashion. SH-SY5Y human neuroblastoma cells carrying a TH-luciferase reporter (SH-SY5Y-THpGL3) were exposed to various ligand (GDNF, NRTN, or ARTN) concentrations and luciferase was measured 18 hours later. Luciferase activity shows a dose-dependent increase (one way ANOVA Newman-Keuls post hoc test n=3; vs control * p<0.05, ** p<0.01, *** p<0.001, error bars=SEM).

FIGS. 4A and 4B are graphs and a Western blot showing that XIB4035 does not induce GFRα/RET receptor signaling. In FIG. 4A, TH-Luciferase transfected SH-SY5Y cells were treated with either 2 nM GDNF or increasing concentrations of XIB4035 either for 10 minutes and then incubated overnight in regular medium or exposed to treatments overnight. Measurements of luciferase activity after the treatments show that GDNF treatment increases TH promoter activity in both conditions, but XIB4035 does not (one way ANOVA Newman-Keuls post hoc test n=3; vs control *** p<0.001, error bars=SEM). In FIG. 4B SH-SY5Y cells were treated with either 2 nM GDNF or various concentrations of XIB4035 for two or 10 minutes and cell were lysed immediately. Anti-phospho-tyrosine Western blot shows that RET phosphorylation (arrow) is induced by GDNF but not by XIB4035.

FIGS. 5A-5D show that XIB4035 potentiates ligand-induced RET signaling. a and b) SH-SY5Y-THpGL3 stable cells were treated with increasing concentrations of GDNF (a) or ARTN (b) with or without 20 μM XIB4035 for 10 minutes. Treatments were washed, cells were maintained overnight in basal medium and then assayed for luciferase activity. For both ligands, XIB4035 co-treatment caused a shift in the non-linear regression of the dose-response curve (FTest: GDNF vs. GDNF+20 μM XIB4035 p=0.00006; ARTN vs. ARTN+20 μM XIB4035 p=0.000005), reduced minimal ligand dose necessary to induce luciferase activity above control (Student's t-test vs. control: GDNF=75 pM (p=0.0063), GDNF+20 μM XIB4035=2.7 pM, (p=0.038), ARTN=75 pM (p=0.0271), ARTN+20 μM XIB4035=2.7 pM, (p=0.0124)), and increased maximal effect (Student's t-test: fold over control: 18 nM GDNF=2.76±0.32 vs. 18 nM GDNF+20 μM XIB4035=3.41±0.41 p=0.0189; 18 nM ARTN=2.78±0.44 vs. 18 nM ARTN+20 μM XIB4035=3.61±0.47, p=0.0241). c and d) SH-SY5Y cells were treated with 2 nM GDNF (c) or 1 nM ARTN (d) with or without 10 or 20 μM XIB4035 for 10 minutes. Cell lysates were either collected immediately or treatment was washed out and replaced with growth media for 30, 60, or 120 minutes. Cell lysates were subjected to phospho-tyrosine Western blot. RET phosphorylation (arrow) in the GDNF or ARTN treated samples returns to baseline between 60 and 120 minutes but is prolonged by addition of XIB4035, e.g. 20 μM XIB4035 prolongs the phosphorylation of RET to at least 120 minutes.

FIG. 6 is a Western blot showing that XIB4035 does not change NGF-induced TrkA phosphorylation. PC12 cells were treated with 50 ng/ml of NGF with or without 20 μM XIB4035 for 10 minutes. Cell lysates were either collected immediately or after cells were incubated in growth media for additional 15, 30, or 45 minutes. Cell lysates were subjected to immunoprecipitation using anti-TrkA antibodies and analysis via phospho-tyrosine Western blot. TrkA phosphorylation is not prolonged by addition of XIB4035.

FIG. 7 shows that RET phosphorylation in Neuro2A (N2A) cells is ligand/GFRα specific. N2A cells, which do not express endogenous GFRαs, were transfected with control (mGFP), GFRα1, or GFRα3 expression constructs and treated with no ligand (Control), 2 nM GDNF, or 1 nM ARTN. No RET phosphorylation was detected in control transfected (mGFP) cells with either GDNF or ARTN treatment. GDNF treatment induced RET phosphorylation only in GFRα1 expressing cells while ARTN only in GFRα3 transfected cells.

FIG. 8 is a Western blot showing that XIB4035 prolongs NRTN/GFRα2-induced RET phosphorylation. B(E)2-C cells, which express GFRα2, were treated under different conditions (no treatment, 2 nM NRTN, or 2 nM NRTN with 20 μM XIB4035) for 10 minutes. Cells were either lysed immediately or after incubation in growth medium for an additional 60 minutes. Phopsho-tyrosine Western blot shows that RET phosphorylation is not detectable at 60 min in the NRTN treated sample, while it is detected in cells co-treated with 20 μM XIB4035.

FIG. 9 shows that XIB4035 treatment prevented slowing of nerve conduction velocity in diabetic mice.

FIG. 10 shows that XIB4035 treatment prevented loss of sub-epidermal fibers in diabetic mice.

### DETAILED DESCRIPTION OF THE INVENTION

As described below, the present invention features the use of XIB4035 for the treatment of large fiber neuropathy, and compositions comprising combinations of XIB4035 and a GFRα ligand (e.g., GDNF, neuturin (NRTN), artemin (ARTN), neublastin, and persephin) and uses thereof for the treatment of neuropathy (e.g., diabetic small fiber neuropathy, injury-associated neuropathy, alcoholism-associated neuropathy, lupus-related neuropathy, HIV-related neuropathy, large fiber neuropathy, a neuropathy associated with chemotherapy, enteric neuropathy).

The invention is based, at least in part, on the surprising discovery that XIB4035 enhances GFRα family receptor signaling in conjunction with ligand stimulation. This discovery is contrary to the conventional thinking in the field, which held that XIB4035 is itself a GFRα1 agonist. This discovery indicates that XIB4035 can be used for the treatment of large fiber neuropathy, and that XIB4035 and other agents described herein can be used in combination with GDNF and other GFR ligands (e.g., neuturin (NRTN), artemin (ARTN), and persephin) to treat neuropathy (e.g., large and small fiber neuropathies, enteric neuropathy).

Small fiber neuropathy (SFN) is a disorder of peripheral nerves commonly found in patients with diabetes mellitus, HIV infection, or those receiving chemotherapy. The complexity of disease etiology has led to a scarcity of effective treatments; however, it has been proposed that target-derived neurotrophic factors may be useful therapeutics. Using two murine models of progressive SFN, over-expression of glial cell line-derived neurotrophic factor (GDNF) in skin keratinocytes or topical application of XIB4035, a reported non-peptidyl agonist of GDNF receptor alpha-1 (GFRα1), were shown to be effective treatments for SFN, preserving and rescuing peripheral sensory fiber structure and function, respectively. Furthermore, results detailed below indicate that XIB4035 is not a GFRα1 agonist, but rather enhances GFRα family receptor signaling in conjunction with ligand stimulation. Taken together, these results indicate that topical application of XIB4035 and other GFRα receptor signaling modulators can be used to treat diabetic neuropathy, and that systemic administration of XIB4035, alone or in combination with GDNF, can be used to treat large fiber neuropathy.

### Neuropathy

Small fiber neuropathy (SFN) is a disorder characterized by degeneration or dysfunction of small diameter unmyelinated nerve fibers (C-fibers) in the peripheral nervous system. Patients with diabetes, HIV infection or undergoing chemotherapy treatments may exhibit a variety of SFN symptoms, including loss of sensation and chronic pain. Despite the prevalence of SFN, its etiology is poorly understood, resulting in a lack of disease-modifying treatments. Reduction in target-derived trophic factor expression has been observed in multiple models of peripheral neuropathy, suggesting insufficient levels of these molecules may underlie the pathogenesis of SFN. Since initial stages of SFN commonly involve nerve terminal degeneration prior to cell death, replenishing or replacing neurotrophic factors promptly after disease onset could potentially be used for treating peripheral neuropathies, as these molecules regulate the survival and function of C-fibers.

One trophic factor necessary for development and survival of a subset of C-fibers is GDNF. GDNF belongs to a family of ligands that interact selectively with different high affinity receptors: Neublastin with GFRα, GDNF with GFRα1, neuturin (NRTN) with GFRα2, artemin (ARTN) with GFRα3, and persephin (PSPN) with GFRα4, although some crosstalk between ligands and receptors has been observed. Neublastin is a neurotrophic factor that binds GFRα3 with high affinity. Neublastin is described, for example, in PCT/EP02/02691 (WO 02/072826), which is incorporated herein by reference. Each ligand/receptor pair forms a complex with the RET receptor tyrosine kinase, leading to its activation and the resulting downstream effects, e.g. tyrosine hydroxylase gene transcription. GDNF family ligands not only play a pivotal role in sensory neuron development, but also appear to be beneficial in the context of peripheral nerve injury. For example, systemic, transgenic or viral delivery of GDNF family members has been shown to attenuate neuropathic symptoms in mouse models of nerve injury. It was hypothesized that topical delivery of GDNF receptor agonists to the skin would be an effective, non-invasive therapeutic approach for progressive SFN. The present study tested whether skin overexpression of GDNF or topical application of XIB4035, a reported non-peptidyl small molecule agonist for GFRα1, could be used to treat two mouse models of progressive SFN arising from different pathogenic mechanisms. Both approaches were effective at preserving nerve structure and function. Furthermore, topical XIB4035 was effective both as a prophylactic treatment when applied before onset of symptoms and a disease modifying therapy when used after SFN onset. Finally, XIB4035 was not a GDNF mimetic as previously reported, but rather, acted as a potentiator of GDNF family receptor function, enhancing ligand-induced signaling. Together, these results indicate that XIB4035 is an effective, non-invasive therapeutic treatment for SFN via modulation of the GFRα/Ret signaling complex.

Prior to this discovery, XIB4035 would not have been systemically administered for the treatment of large fiber neuropathy because XIB4035 was thought to act as a GDNF mimetic. The systemic administration of GDNF has been shown to adversely affect patients suffering from Parkinson's disease. The finding that XIB4035 simply enhances GDNF activity, rather than acting as a GFRα agonist indicates that XIB4035 could be safely and effectively administered systemically for the treatment of large fiber neuropathy because as an enhancer XIB4035 only increases activity when both GDNF and the enhancer are present. In contrast, the exogenous administration of GDNF provides high levels of GDNF throughout the body, which may result in the promiscuous activation of receptors that would not normally be exposed to endogenous GDNF. Thus, the invention further for the treatment of large fiber neuropathies, which can affect sensory neurons, motor neurons, or both, as well as for the treatment of enteric neuropathy (e.g., diabetic enteric neuropathy).

In contrast to small fiber neuropathies, large fiber neuropathies manifest with the loss of joint position and vibration sense and sensory ataxia. The diagnosis of large fiber neuropathy is made using methods known to physicians skilled in the art of neurological testing. Electrodiagnostic (EDx) tests include sensory, motor nerve conduction, F response, H reflex and needle electromyography (EMG). Clinical indicators can also be used to diagnose a large fiber neuropathy. Clinical indicators include weakness, pain, and problems with gait or other movement. EDx helps in documenting the extent of sensory motor deficits, categorizing demyelinating (prolonged terminal latency, slowing of nerve conduction velocity, dispersion and conduction block) and axonal (marginal slowing of nerve conduction and small compound muscle or sensory action potential and dennervation on EMG).

### Pharmaceutical Therapeutics and Therapeutic Methods

As reported herein, XIB4035 has now been identified as enhancing GDNF activity. Thus, other agents identified as enhancing the expression or activity of a GDNF polypeptide are useful for preventing or ameliorating a neuropathy. In one therapeutic approach, an agent identified as described herein is administered to the site of a potential or actual disease-affected tissue or is administered systemically. The dosage of the administered agent depends on a number of factors, including the size and health of the individual patient. For any particular subject, the specific dosage regimes should be adjusted over time according to the individual need and the professional judgement of the person administering or supervising the administration of the compositions.

Other agents useful for the treatment of neuropathy (e.g., diabetic small fiber neuropathy, injury-associated neuropathy, alcoholism-associated neuropathy, lupus-related neuropathy, HIV-related neuropathy, large fiber neuropathy, a neuropathy associated with chemotherapy, or enteric neuropathy), alone or in combination with GDNF, include one or more of the following:

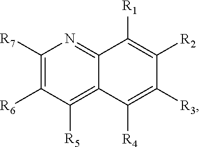

wherein R₁-R₇ are each independently H, hydroxy, halogen, substituted or unsubstituted alkyl, substituted or unsubstituted alkenyl, substituted or unsubstituted alkynyl, substituted or unsubstituted arylalkyl, substituted or unsubstituted amine, substituted or unsubstituted alkylamine, substituted or unsubstituted dialkylamine, substituted or unsubstituted alkoxy, substituted or unsubstituted aryl, substituted or unsubstituted heteroaryl, substituted or unsubstituted alkoxy, substituted or unsubstituted haloalkyl, or a pharmaceutically acceptable salt thereof, under conditions effective to treat or prevent the peripheral neuropathy in the subject.

The invention encompasses all alternative combinations of particular embodiments: R₂ is halogen, particularly Cl;

R₅ is a substituted amine, particularly optionally-substituted alkyl substituted secondary amine, particularly wherein the alkyl is substituted with a dialkylamine such as 1-methyl-3-diethylaminobutyl, 1-methyl-4-dimethylaminobutyl, 1-ethyl-4-dimethylaminobutyl, 1-ethyl-4-diethylaminobutyl, or 1-methyl-4-diethylaminobutyl:

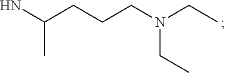

R₇ is a substituted alkenyl, particularly optionally-substituted phenyl substituted ethenyl, particularly such as

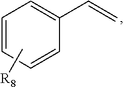

and/or
wherein R₈ is hydrogen or halogen, such as Cl, particularly ortho-chloro:
such as wherein the compound has formula:

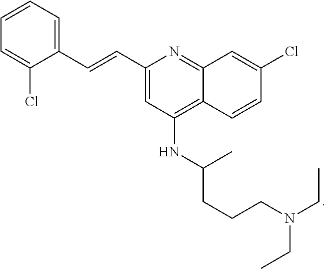 [SMILES: CCN(CC)CCCC(C)Nc1cc(/C=C/c2ccccc2Cl)nc2cc(Cl)ccc12]

In another aspect, the invention provides methods and compositions for treating or preventing a small or large fiber peripheral neuropathy in a subject determined to be in need thereof, and generally comprising: administering to the subject an agent that enhances the expression or activity of GDNF (e.g., XIB4035).

In one embodiment, the invention provides a compound of the formula:

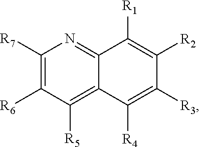

wherein R₁-R₇ are each independently H, hydroxy, halogen, substituted or unsubstituted alkyl, substituted or unsubstituted alkenyl, substituted or unsubstituted alkynyl, substituted or unsubstituted arylalkyl, substituted or unsubstituted amine, substituted or unsubstituted alkylamine, substituted or unsubstituted dialkylamine, substituted or unsubstituted alkoxy, substituted or unsubstituted aryl, substituted or unsubstituted heteroaryl, substituted or unsubstituted alkoxy, substituted or unsubstituted haloalkyl, or a pharmaceutically acceptable salts thereof.

“Alkyl” as used herein refers to a saturated hydrocarbon radical which may be straight-chain or branched-chain (for example, ethyl, isopropyl, t-amyl, or 2,5-dimethylhexyl) or cyclic (for example cyclobutyl, cyclopropyl or cyclopentyl) and contains from 1 to 24 carbon atoms. This definition applies both when the term is used alone and when it is used as part of a compound term, such as “haloalkyl” and similar terms. In some embodiments, preferred alkyl groups are those containing 1 to 4 carbon atoms, which are also referred to as “lower alkyl.” In some embodiments preferred alkyl groups are those containing 5 or 6 to 24 carbon atoms, which may also be referred to as “higher alkyl”.

“Alkenyl,” as used herein, refers to a straight or branched chain hydrocarbon containing from 2 to 24 carbons and containing at least one carbon-carbon double bond formed by the removal of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. “Lower alkenyl” as used herein, is a subset of alkenyl and refers to a straight or branched chain hydrocarbon group containing from 1 to 4 carbon atoms.

“Alkynyl,” as used herein, refers to a straight or branched chain hydrocarbon group containing from 2 to 24 carbon atoms and containing at least one carbon-carbon triple bond. Representative examples of alkynyl include, but are not limited, to acetylenyl, 1-propynyl, 2-propynyl, 3-butynyl, 2-pentynyl, 1-butynyl and the like. “Lower alkynyl” as used herein, is a subset of alkyl and refers to a straight or branched chain hydrocarbon group containing from 1 to 4 carbon atoms.

“Alkoxy” refers to an alkyl radical as described above which also bears an oxygen substituent which is capable of covalent attachment to another hydrocarbon radical (such as, for example, methoxy, ethoxy and t-butoxy).

“Alkylthio” as used herein refers to an alkyl group, as defined herein, appended to the parent molecular moiety through a thio moiety, as defined herein. Representative examples of alkylthio include, but are not limited, methylthio, ethylthio, tert-butylthio, hexylthio, and the like.

“Aryl” or “aromatic ring moiety” refers to an aromatic substituent which may be a single ring or multiple rings which are fused together, linked covalently or linked to a common group such as an ethylene or methylene moiety. The aromatic rings may each contain heteroatoms and hence “aryl” encompasses “heteroaryl” as used herein. Representative examples of aryl include, azulenyl, indanyl, indenyl, naphthyl, phenyl, tetrahydronaphthyl, biphenyl, diphenylmethyl, 2,2-diphenyl-1-ethyl, thienyl, pyridyl and quinoxalyl. “Aryl” means substituted or unsubstituted aryl unless otherwise indicated and hence the aryl moieties may be optionally substituted with halogen atoms, or other groups such as nitro, carboxyl, alkoxy, phenoxy and the like. Additionally, the aryl radicals may be attached to other moieties at any position on the aryl radical which would otherwise be occupied by a hydrogen atom (such as, for example, 2-pyridyl, 3-pyridyl and 4-pyridyl).

“Heteroaryl” means a cyclic, aromatic hydrocarbon in which one or more carbon atoms have been replaced with heteroatoms. If the heteroaryl group contains more than one heteroatom, the heteroatoms may be the same or different. Examples of heteroaryl groups include pyridyl, pyrimidinyl, imidazolyl, thienyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, and benzo[b]thienyl. Preferred heteroaryl groups are five and six membered rings and contain from one to three heteroatoms independently selected from O, N, and S. The heteroaryl group, including each heteroatom, can be unsubstituted or substituted with from 1 to 4 substituents, as chemically feasible.

“Halo” or “halogen,” as used herein, refers to —Cl, —Br, —I or —F.

“Haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, 2-chloro-3-fluoropentyl, and the like.

“Hydroxy,” as used herein, refers to an —OH group.

“Amine” or “amino” as used herein, refers to a nitrogen atom attached by single bonds to hydrogen atoms, alkyl groups, aryl groups, or a combination of these three. An organic compound that contains an amino group is called an amine Amines are derivatives of the inorganic compound ammonia, NH₃. When one, two, or all three of the hydrogens in ammonia are replaced by an alkyl or aryl group, the resulting compound is known as a primary, secondary, or tertiary amine, respectively.

In certain embodiments, R₂ may be halogen, R₅ may be a substituted amine, and/or R₇ may be a substituted alkenyl such as

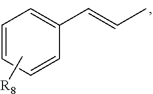

wherein R₈ may be H or halogen, for example, Cl.

In preferred embodiments, R₂ is Cl, R₅ is

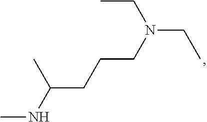

### and R₇ is

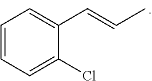

Wuinoline compounds described herein are commercially available and/or readily produced using convention organic synthesis. Relevant derivitization schemes are known in the art, such as described in “Synthesis of substituted 4-(δ-diethylamino-α-methylbutylamino)-2-styrylquinolines”, Berenfel'd, V. M.; Yakhontov, L. N.; Yanbukhtin, N. A.; Krasnokutskaya, D. M.; Vatsenko, S. V.; Rubtsov, M. V. Zhurnal Obshchei Khimii (1962), 32 2169-77. CODEN: ZOKHA4 ISSN: 0044-460X; “Syntheses in the isoquinoline series. Hofmann degradation of 1-phenyl-substituted 1,2,3,4-tetrahydroisoquinolines,” Rheiner, A., Jr.; Brossi, A. F. Hoffmann-La Roche & Co., A.-G., Basel, Switz. Helvetica Chimica Acta (1962), 45 2590-600. CODEN: HCACAV ISSN: 0018-019X; “Synthesis and antileishmaniasis activity of 2-(2′-chlorostyryl)-4-(δ-diethylamino-α-methylbutylamino)-7-chloroquinazoline diphosphate,” Yakhontov, L. N.; Zhikhareva, G. P.; Mastafanova, L. I.; Evstratova, M. I.; Pershin, G. N.; Moskalenko, N. Yu.; Pushkina, T. V.; Kutchak, S. N.; Fadeeva, N. I.; et al. VNIFI, Moscow, USSR. Khimiko-Farmatsevticheskii Zhurnal (1987), 21(1), 38-49. CODEN: KHFZAN ISSN: 0023-1134; and “Reaction products of 4-[[4-(diethylamino)-1-methylbutyl]amino]-7-chloroquinaldine with o-chlorobenzaldehyde,” Uritskaya, M. Ya.; Anisimova, O. S.; Tubina, I. S.; Vinokurova, T. Yu.; Pershin, G. N.; Moskalenko, N. Yu.; Gus'kova, T. A.; Kutchak, S. N.; Stebaeva, L. F. Vses. Nauchno-Issled. Khim.-Farm. Inst., Moscow, USSR. Khimiko-Farmatsevticheskii Zhurnal (1983), 17(11), 1334-40. CODEN: KHFZAN ISSN: 0023-1134.

Anti-peripheral neuropathic activity is readily confirmed in topical formulations and the convenient animal models, as demonstrated below. The subject compounds are topically-active, antineuropathic quinolines, particularly aminoquinolines, particularly 4- and 8-aminoquinolines, particularly chloroquines (chloroquine and derivatives thereof), and include compounds of Tables 1-3:

TABLE 1

| | |
|---|---|
| 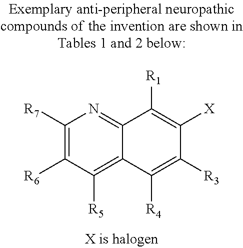 | 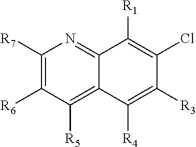 |
| 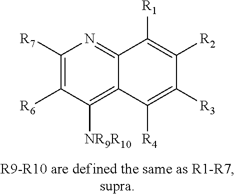 | 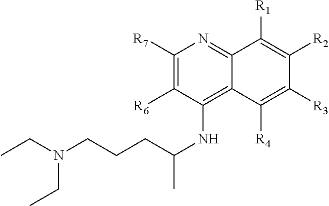 |
| 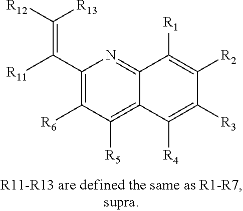 | 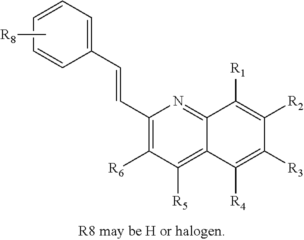 |
| 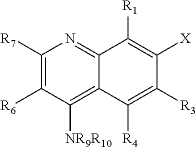 | 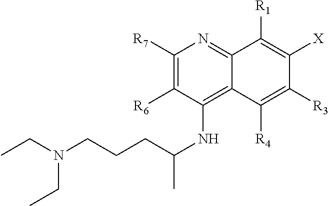 |
| 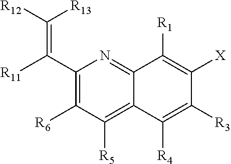 | 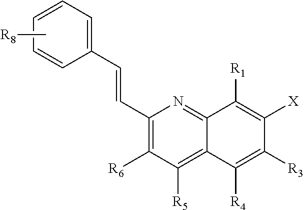 |
| 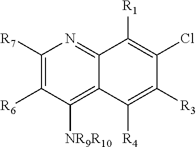 | 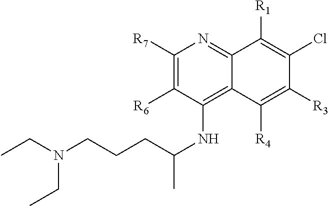 |
| 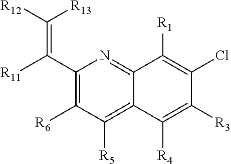 | 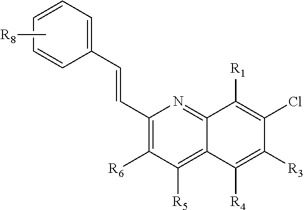 |
| 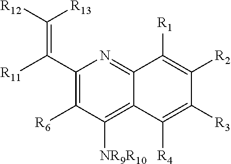 | 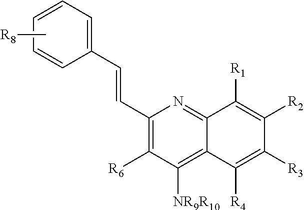 |
| 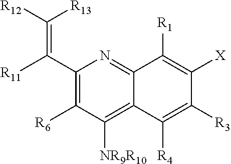 | 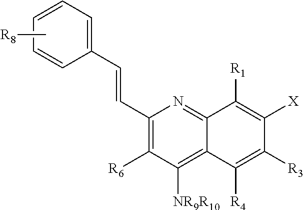 |
| 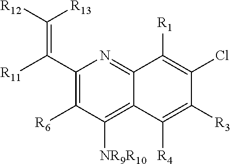 | 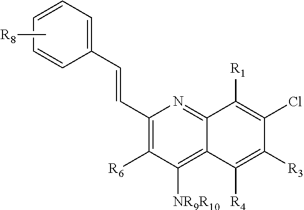 |
| 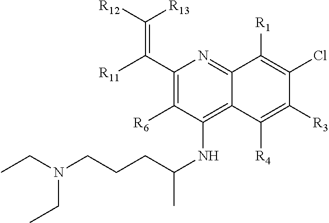 | 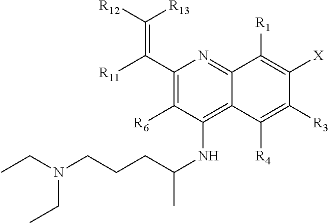 |
| 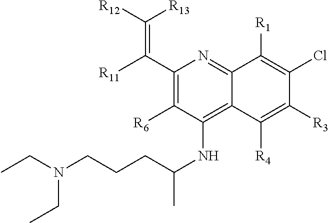 | 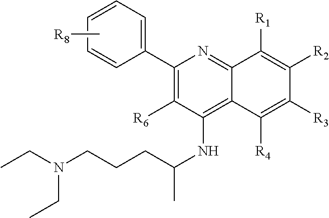 |
| 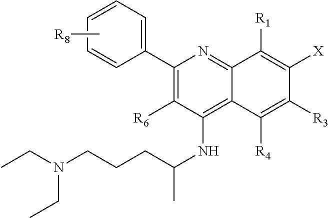 | 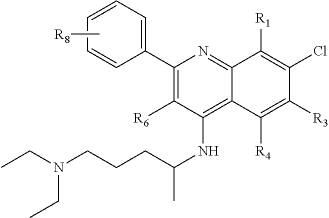 |
| 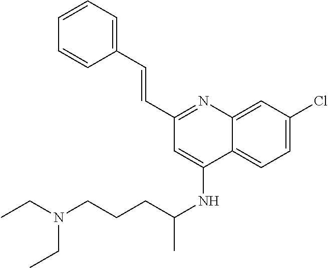 [SMILES: CCN(CC)CCCC(C)Nc1cc(/C=C/c2ccccc2)nc2cc(Cl)ccc12] | 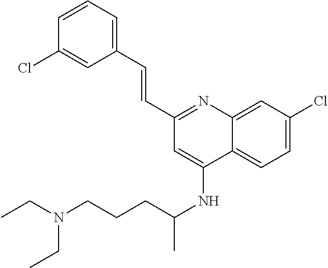 [SMILES: CCN(CC)CCCC(C)Nc1cc(/C=C/c2cccc(Cl)c2)nc2cc(Cl)ccc12] |
| 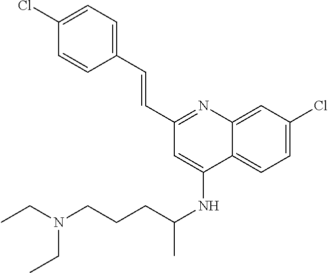 [SMILES: CCN(CC)CCCC(C)Nc1cc(/C=C/c2ccc(Cl)cc2)nc2cc(Cl)ccc12] | 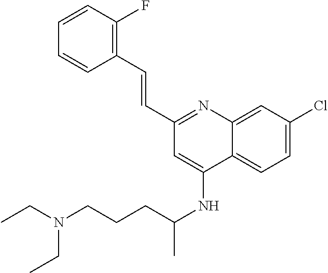 [SMILES: CCN(CC)CCCC(C)Nc1cc(/C=C/c2ccccc2F)nc2cc(Cl)ccc12] |
| 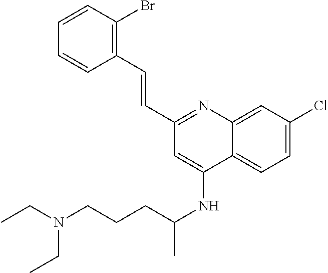 [SMILES: CCN(CC)CCCC(C)Nc1cc(/C=C/c2ccccc2Br)nc2cc(Cl)ccc12] | 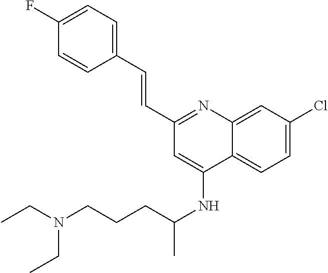 [SMILES: CCN(CC)CCCC(C)Nc1cc(/C=C/c2ccc(F)cc2)nc2cc(Cl)ccc12] |
| 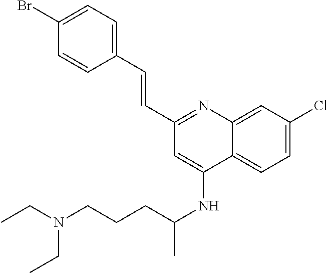 [SMILES: CCN(CC)CCCC(C)Nc1cc(/C=C/c2ccc(Br)cc2)nc2cc(Cl)ccc12] | 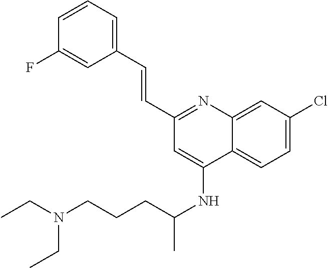 [SMILES: CCN(CC)CCCC(C)Nc1cc(/C=C/c2cccc(F)c2)nc2cc(Cl)ccc12] |
| 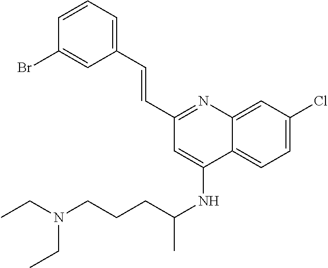 [SMILES: CCN(CC)CCCC(C)Nc1cc(/C=C/c2cccc(Br)c2)nc2cc(Cl)ccc12] | 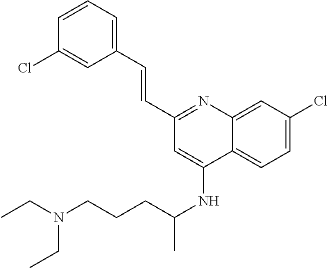 [SMILES: CCN(CC)CCCC(C)Nc1cc(/C=C/c2cccc(Cl)c2)nc2cc(Cl)ccc12] |

TABLE 2

| | |
|---|---|
| 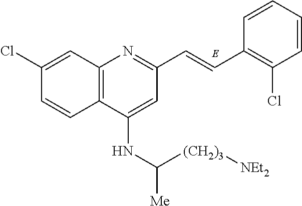 | 1,4-Pentanediamine, N4-[7-chloro-2-[(1E)-2-(2- chlorophenyl)ethenyl]-4-quinolinyl]-N1,N1- diethyl- |
| 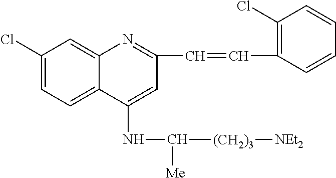 | 1,4-Pentanediamine, N4-[7-chloro-2-[2-(2- chlorophenyl)ethenyl]-4-quinolinyl]-N1,N1- diethyl- |
| 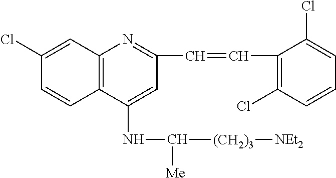 | 1,4-Pentanediamine, N4-[7-chloro-2-[2-(2,6- dichlorophenyl)ethenyl]-4-quinolinyl]-N1,N1- diethyl- |
| 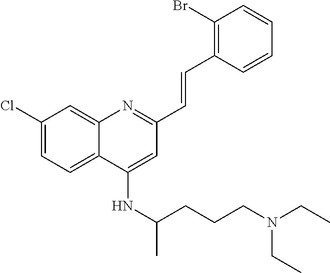 [SMILES: CCN(CC)CCCC(C)Nc1cc(/C=C/c2ccccc2Br)nc2cc(Cl)ccc12] | 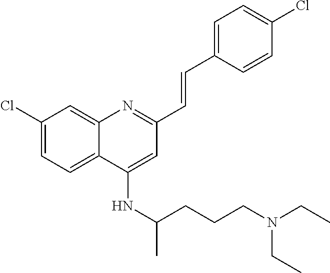 [SMILES: CCN(CC)CCCC(C)Nc1cc(/C=C/c2ccc(Cl)cc2)nc2cc(Cl)ccc12] |
| 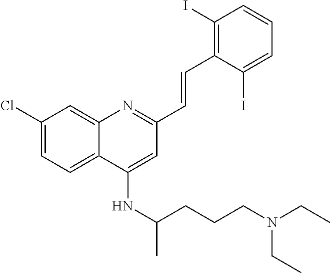 [SMILES: CCN(CC)CCCC(C)Nc1cc(/C=C/c2c(I)cccc2I)nc2cc(Cl)ccc12] | 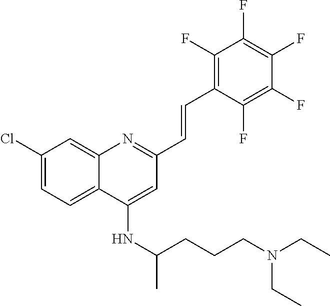 [SMILES: CCN(CC)CCCC(C)Nc1cc(/C=C/c2c(F)c(F)c(F)c(F)c2F)nc2cc(Cl)ccc12] |
| 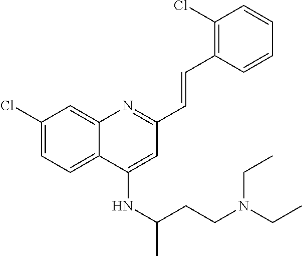 [SMILES: CCN(CC)CCC(C)Nc1cc(/C=C/c2ccccc2Cl)nc2cc(Cl)ccc12] | 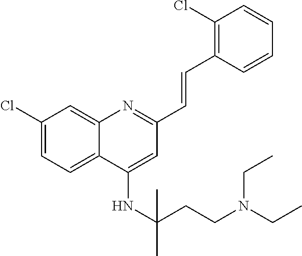 [SMILES: CCN(CC)CCC(C)(C)Nc1cc(/C=C/c2ccccc2Cl)nc2cc(Cl)ccc12] |

TABLE 3
7-chloro-4-(4-diethylamino-1-methylbutylamino)quinoline (chloroquine);
7-hydroxy-4-(4-diethylamino-1-methylbutylamino)quinoline; chloroquine phosphate;
7-chloro-4-(4-diethylamino-1-butylamino)quinoline (desmethylchloroquine);
7-hydroxy-4-(4-diethylamino-1-butylamino)quinoline;
7-chloro-4-(1-carboxy-4-diethylamino-1-butylamino)quinoline;
7-hydroxy-4-(1-carboxy-4-diethylamino-1-butylamino)quinoline;
7-chloro-4-(1-carboxy-4-diethylamino-1-methylbutylamino)quinoline;
7-hydroxy-4-(1-carboxy-4-diethylamino-1-methylbutylamino) quinoline;
7-chloro-4-(4-ethyl-(2-hydroxyethyl)-amino-1-methylbutylamino)quinoline
(hydroxychloroquine);
7-hydroxy-4-(4-ethyl-(2-hydroxyethyl)-amino-1-methylbutyl amino)quinoline;
hydroxychloroquine phosphate;
7-chloro-4-(4-ethyl-(2-hydroxyethyl)-amino-1-butylamino)quinoline
(desmethylhydroxychloroquine);
7-hydroxy-4-(4-ethyl-(2-hydroxyethyl)-amino-1-butylamino) quinoline;
7-chloro-4-(1-carboxy-4-ethyl-(2-hydroxyethyl)-amino-1-butylamino)quinoline;
7-hydroxy-4-(1-carboxy-4-ethyl-(2-hydroxyethyl)-amino-1-butylamino)quinoline;
7-chloro-4-(1-carboxy-4-ethyl-(2-hydroxyethyl)-amino-1-
methylbutylamino)quinoline;
7-hydroxy-4-(1-carboxy-4-ethyl-(2-hydroxyethyl)-amino-1-
methylbutylamino)quinoline;
8-[(4-aminopentyl)amino]-6-methoxydihydrochloride quinoline;
1-acetyl-1,2,3,4-tetrahydroquinoline; 8-[4-aminopentyl)amino]-6-methoxyquinoline
dihydrochloride;
1-butyryl-1,2,3,4-tetrahydroquinoline;
7-chloro-2-(o-chlorostyryl)-4-[4-diethylamino-1-methylbutyl] aminoquiinoline
phosphate;
3-chloro-4-(4-hydroxy-α,α′-bis(2-methyl-1-pyrrolidinyl)-2,5-xylidinoquinoline, 4-
[(4-diethylamino)-1-methylbutyl)amino]-6-methoxyquinoline;
3,4-dihydro-1 (2H)-quinolinecarboxyaldehyde;
1,1′-pentamethylenediquinoleinium diiodide;
8-quinolinol sulfate;
Chloroquine 4-acetaminosalicylate;
Chlorquinaldol;
3-Methylchloroquine;
3-Carboxy-4-hydroxy-7-chloroquinoline;
4,7-Dichloroquinoline;
7-Chloro-4-hydroxyquinoline;
6-Chloroquinaldine;
N,2,6-Trichloro-4-benzoquinone imine;
Hydroxychloroquine;
Chloranil;
Clioquinol;
Cloxyquin;
Chloroquine sulfate;
8-Chloroquinoline;
4-Chloroquinoline;
3-Chloroquinoline;
6-Chloroquinoline;
2-Chloroquinoline;
2-Chloro-1,4-hydroxyquinone;
5-Chloroquinoline;
2-Chloro-1,4-benzoquinone;
2,6-Dichlorobenzoquinone;
Hydroxychloroquine sulfate;
Chloroxine;
7-Chloroquinolin-8-ol;
Chloroquinine phosphate;
2-Chloroquinoxaline;
Desethylchloroquine;
2,3-Dichloroquinoxaline-6-carbonylchloride;
2,3-Dichloroquinoxaline;
2-Chloroquinoline-4-carbonyl chloride;
4,11-Dichloroquinacridonequinone;
2,9-Dichloroquino(2,3-b)acridine-6,7,13,14(5H,12H)-tetrone;
2,3,6-Trichloroquinoxaline;
Chlorquinox;
Chloroquine hydrochloride;
Glafenine;
Chloroquine mustard;
N,N-Dideethylchloroquine;
Cletoquine;
Chloroquine-ethyl phenyl mustard;
4-Chloroquinazoline;
4-(3′,5′-Bis(pyrrolidinomethyl)-4-hydroxyanilino)-7-chloroquinoline;
6-Chloroquinoxaline;
6-Chloro-8-aminoquinoline;
2-Chloromethyl-4-phenyl-6-chloroquinazoline-3-oxide;
2-Chloroquinazoline;
4-(2-Methyl-1-pyrrolidyl)-7-chloroquinoline;
6,7-Dichloroquinoline-5,8-dione;
6,7-Dichloroquinoxaline-2,3-dione;
Cloquinate;
8-Quinolinol, 7-bromo-5-chloro-;
Collagenan;
Dichlorquinazine;
4,7-Dichloroquinolinium tribromide;
Chloroquinoline;
Chloroquine diorotate;
2,4,6-Triamino-5-chloroquinazoline;
Methyl-8-(5,7-dichloroquinolyl)carbonic acid ester;
6-Amino-7-chloro-5,8-dioxoquinoline;
4,8-Dichloroquinoline;
5-Chloroquinolin-8-ol hydrochloride;
3-Phenyl-4-hydroxy-7-chloroquinolin-2(1H)-one;
N-Methyl-6-chloroquinolinium iodide;
3-Chloroquinuclidine hydrochloride;
Halacrinate;
1-Phenacyloxime-4,5-dichloroquinolinium chloride hydrate;
Chloroquine diascorbate;
2-(7-Chloroquinolin-4-yl)anthranilic acid hydrochloride;
Tripiperaquine;
2-(2-Chlorostyryl)-4-(delta-diethylamino-alpha-methylbutylamino)-7-
chloroquinazoline;
(+)-Chloroquine;
(−)-Chloroquine;
7-Chloro-4-(3-octylaminopropyl)aminoquinoline 1-oxide;
Ethyl chloroquine mustard;
L-Chloroquine;
2,6-Dianilino-6-chloroquinoxaline;
2-(2-(5-Nitrofuryl)vinyl)-4-(delta-diethylamino-alpha-methylbutylamino)-7-
chloroquinazoline;
D-Chloroquine;
2,3-Bis(allylamino)-6-chloroquinoxaline;
7-Chloroquinolin-4-ol hydrochloride;
2-Amino-3,4-dichloroquinoline;
Quizalofop;
Presocyl;
Tris(5,7-dichloroquinolin-8-olato-N1,O8)aluminium;
Contramibial;
Quinclorac;
N-(4-((7-Chloroquinolin-4-yl)amino)pentyl)-N-ethylacetamide;
7-Bromo-5-chloroquinolin-ol;
Chlorsulfaquinoxaline;
1-Dimethylaminopropyl-3-methyl-6-chloroquinoxaline-2(1H)-one;
Propaquizafop;
3-Chloroquinoline-8-carboxylic acid;
5,10,15,20-Tetraphenyl-1-3-(4-(4-aminobutyl)-7-
chloroquinoline)propioamidoporphine;
4-((Carboxymethyl)amino)-5,7-dichloroquinoline-2-carboxylic acid;
4-((Carboxymethyl)oxy)-5,7-dichloroquinoline-2-carboxylic acid;
5,7-Dichlorokynurenic acid;
N1,N2-Bis(7-chloroquinolin-4-yl)cyclohexane-1,2-diamine;
Meclinertant;
5-(2-(1-(3-(2-(7-Chloroquinolin-2-yl)ethenyl)benzyl)indol-7-yl)ethyl)-1H-tetrazole;
(N1-(7-Chloroquinolin-4-yl)-3-(N3,N3-diethylamino)propylamine) dihydrochloride
trihydrate; and
enantiomers thereof, and mixtures thereof, and suitable pharmaceutical salts thereof.

Use of N4-{7-chloro-2-[(E)-2-(2-chloro-phenyl)-vinyl]-quinolin-4-yl}-N1,N1-diethyl-pentane-1,4-diamine (XIB4035), also known as 7-chloro-2-(o-chlorostyryl)-4-[4diethylamino-1-methylbutyl]aminoquinoline phosphate), and 2-(2-Chlorostyryl)-4-(delta-diethylamino-alpha-methylbutylamino)-7-chloroquinazoline (CAS RN 57942-32-2; CAS 10023-54-8) is described, for example, by Tokugawa et al., Neurochem Intnl 2003, 42, 81-86; WO01003649; and JP 2008-230974.

For therapeutic uses, the compositions or agents disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neuropathy. Generally, amounts will be in the range of those used for other agents used in the treatment of other diseases associated with neuropathy, although in certain instances lower amounts will be needed because of the increased specificity of the compound. A compound is administered at a dosage that enhances GDNF activity.

### Formulation of Pharmaceutical Compositions

The administration of a compound for the treatment of a neuropathy (e.g., diabetic neuropathy, small fiber neuropathy, injury-associated neuropathy, alcoholism-associated neuropathy, lupus-related neuropathy, HIV-related neuropathy, large fiber neuropathy, a neuropathy associated with chemotherapy, or enteric neuropathy) may be by any suitable means that results in a concentration of the therapeutic that, combined with other components, is effective in ameliorating, reducing, or stabilizing a neuropathy. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neuropathy by using carriers or chemical derivatives to deliver the therapeutic agent to a particular cell type (e.g., peripheral neuron, large fiber neuron, motor neuron, sensory neuron). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a neuropathy, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active inflammatory bowel disorder therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms for Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active therapeutic substance). The coating may be applied on the solid dosage form in a similar manner as that described in Encyclopedia of Pharmaceutical Technology, supra.

At least two neuropathy therapeutics may be mixed together in the tablet, or may be partitioned. In one example, the first active neuropathy therapeutic is contained on the inside of the tablet, and the second active neuropathy therapeutic is on the outside, such that a substantial portion of the second active neuropathy therapeutic is released prior to the release of the first active neuropathy therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active neuropathy therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Polynucleotide Therapy

The invention provides methods for recombinantly expressing GDNF or another GFRα ligand in a cell, tissue, or organ. If desired, a viral vector (e.g., an adeno-associated viral vector) is used to inducibly or constitutively express a GFRα ligand polypeptide. Polynucleotide therapy featuring a polynucleotide (e.g., an AAV expression vector, such as an AAV-2, AAV-9 vector) encoding a GFRα ligand protein, variant, or fragment thereof is one therapeutic approach for treating neuropathy. Such GFRα ligand-expressing nucleic acid molecules can be delivered to cells (e.g., skin, epithelial cells, muscle, myocytes, nerves, blood vessel, endothelial cells) of a subject having neuropathy. The polynucleotide encoding a GFRα ligand protein must be delivered to the cells of a subject in a form in which they can be taken up so that therapeutically effective levels of GFRα ligand can be produced. Preferably, persistent expression of an GFRα ligand polypeptide is maintained at an effective level for longer than 1 week, 2 weeks, 3 weeks, or longer than 1, 3, 6, or 12 months. If desired, the expression of GFRα ligand is combined with any standard method of treating neuropathy.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a GFRα ligand polypeptide, variant, or fragment thereof, can be cloned into a retroviral vector and expression can be driven from its endogenous promoter, from a retroviral long terminal repeat, or from a promoter specific for a target cell type of interest (e.g., muscle, skin, neurons, endothelial cells).

In particular embodiments, the following promoters may be used: Glial fibrillary acidic protein (GFAP) promoter, CMV (cytomegalovirus) promoter, CAG (chicken b-actin promoter, Neuron-specific promotors, such as 1.8 kb neuron-specific enolase promoter.

In other embodiments, any of the following vectors may be used: Adeno-associated viral vector (AAV), lentiviral vector, retroviral vector, herpes simplex viral vector and any vector that can infect brain cells. More specifically, vectors useful in the methods of the invention include a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In one embodiment, an adeno-associated viral vector (e.g., serotype 2, 9) is used to administer a polynucleotide intravenously, into the cerebrospinal fluid, or by surgical injection into the brain.

Non-viral approaches can also be employed for the introduction of a therapeutic to a cell of a patient requiring treatment or prevention of neuropathy. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). In one embodiment, the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue or delivered via a canula.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types (e.g. endothelial cells, neurons, astrocytes, glia) can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant GFRα ligand variant, or fragment thereof, either directly to the site of a potential or actual disease-affected tissue, to an organ where the polypeptide will have a therapeutic effect, or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

### GFRα Ligand Therapeutics

For therapeutic uses, a viral expression vector comprising a polynucleotide encoding a GFRα ligand polypeptide disclosed herein may be administered systemically, for example, formulated in a pharmaceutically-acceptable buffer, such as physiological saline. Preferable routes of administration include, for example, intravenous, intra-arterial, intramuscular, subcutaneously, intradermal, intrathecal, into the cerebrospinal fluid, into the ventricles of the brain, or any other injection site that provides continuous, sustained levels of expression in the patient to treat a neuropathy.

Treatment of human patients or other animals will be carried out using a therapeutically effective amount of a nucleic acid molecule or polypeptide therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the cellular deficiency. Generally, amounts will be in the range of those used for other therapeutic polypeptide or protein therapy agents used in the treatment of other diseases. In one embodiment, polypeptides of the invention are administered at a dosage that controls the clinical or physiological symptoms of neuropathy as determined by a diagnostic method known to one skilled in the art.

### Formulation of Pharmaceutical Compositions

The administration of a composition of the invention for the treatment of a neuropathy may be by any suitable means that results in expression of an effective amount of GFRα ligand that, combined with other components, is effective in ameliorating, reducing, or stabilizing the disease. For example, an amount that reduces neuropathy. A therapeutic GFRα ligand expression vector or GFRα ligand polypeptide may be contained in any appropriate amount in any suitable carrier substance, and is generally present in an amount of 1-95% by weight of the total weight of the composition. The composition may be provided in a dosage form that is suitable for parenteral (e.g., intravenously, intra-arterial) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

If desired, therapeutic compositions of the invention (e.g., a viral expression vector comprising a polynucleotide encoding a GFRα ligand polypeptide) are provided together with other agents that are useful for reducing the symptoms of or that are otherwise therapeutic for neuropathy.

### Methods of Delivery

A pharmaceutical composition comprising a viral expression vector comprising a polynucleotide encoding an GFRα ligand polypeptide may be administered by injection (intravenous, intra-arterial, intra-spinal, intra-ventricular or the like), infusion or implantation in dosage forms, formulations, or via suitable delivery devices or implants containing conventional, non-toxic pharmaceutically acceptable carriers and adjuvants. In one embodiment, a therapeutic composition of the invention is provided via an osmotic pump. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added. The composition may be in the form of a solution, a suspension, an emulsion, an infusion device, or a delivery device for implantation, or it may be presented as a dry powder to be reconstituted with water or another suitable vehicle before use. Apart from the active GFRα ligand polynucleotide therapeutic(s), the composition may include suitable parenterally acceptable carriers and/or excipients. The active GFRα ligand polynucleotide therapeutic (s) may be incorporated into an osmotic pump, microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in a form suitable for sterile injection. To prepare such a composition, the suitable active GFRα ligand polynucleotide therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

In one embodiment, a therapeutic composition of the invention (e.g., GFRα ligand polypeptide, an expression vector comprising a polynucleotide encoding an GFRα ligand polypeptide, or cell comprising such agents) is provided locally via a canula. For example, for delivery to cells surrounding a neuropathy, a composition of the invention is provided via an artery or other vessel supplying blood to the neuropathy. In another embodiment, an GFRα ligand expression vector is administered via a ventricle in fluid communication with the neuropathy or surrounding cells. In another embodiment, an GFRα ligand expression vector is administered to the cerebrospinal fluid of a subject. In other embodiments, a composition of the invention is provided via an osmotic pump. Desirably, the osmotic pump provides for the controlled release of the composition over 1-3 days, 3-5 days, 5-7 days, or for 2, 3, 4, or 5 weeks.

### Combination Therapies

Compositions of the invention may, if desired, be delivered in combination with any other therapeutic known in the art. In one embodiment, a GFRα ligand expression vector of the invention is used to reduce neuropathy in a subject. Therapeutic efficacy does not require elimination of the neuropathy. Therapeutic efficacy is achieved if the methods of the invention reduce the symptoms of neuropathy, increase neuronal function as measured in electrodiagnostic testing, or that enhance neuronal survival. Desirably, this reduction in neuropathy is by at least about 5, 10, or 15%, more desirably by at least about 20%, 25%, or even by 30%, or even more desirably by 50%, 75%, 85% or more a reduction in symptoms of neuropathy or an increase in function.

### Kits or Pharmaceutical Systems

The present compositions may be assembled into kits or pharmaceutical systems for use in ameliorating neuropathy. In one embodiment, the kit comprises a GFRα ligand expression vector and instructions for the use of the vector. Kits or pharmaceutical systems according to this aspect of the invention comprise a carrier means, such as a box, carton, tube or the like, having in close confinement therein one or more container means, such as vials, tubes, ampules, bottles and the like. The kits or pharmaceutical systems of the invention may also comprise associated instructions for using the agents of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Overexpression of GDNF by Keratinocytes Prevents Progressive SFN

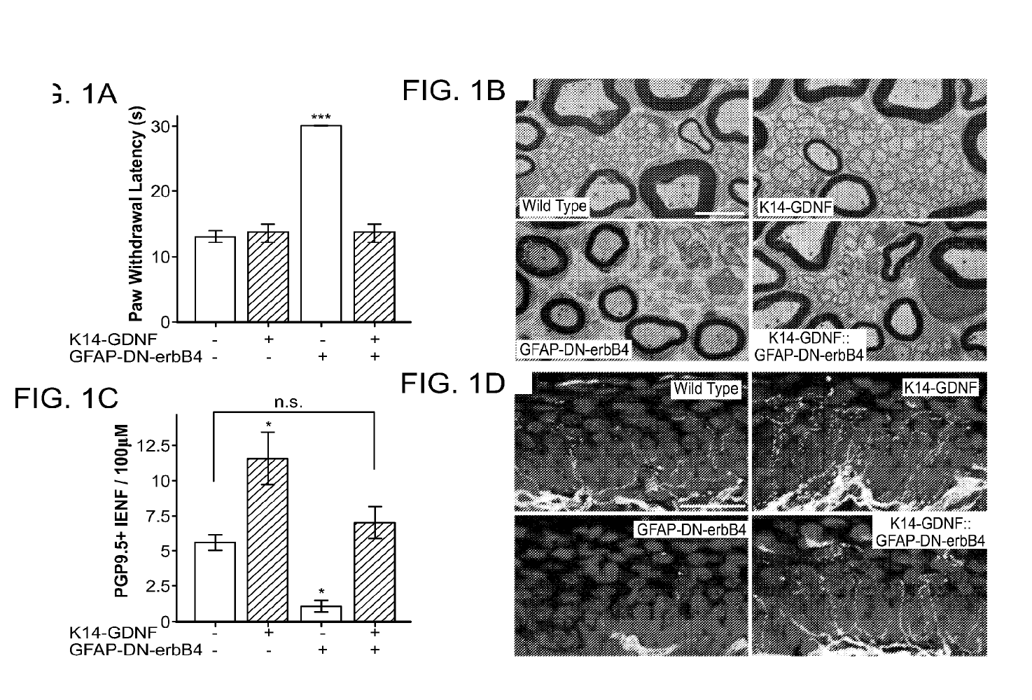

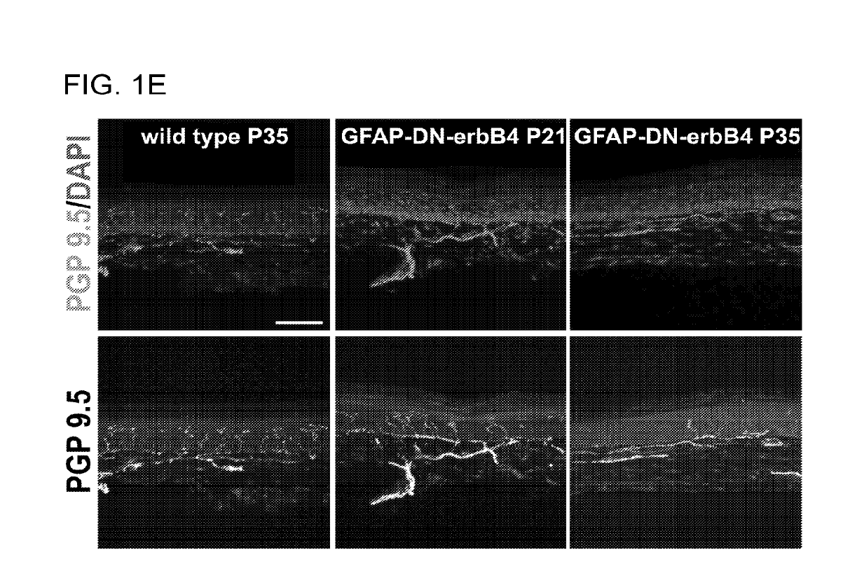

Transgenic mice in which ErbB receptor function has been eliminated in non-myelinating cells (NMSCs) (GFAP-DN-erbB4) develop SFN, including loss of thermal nociception, breakdown of Remak bundles (multiple c-fibers ensheathed by one NMSC), and degeneration of C-fibers (Chen et al. Nat Neurosci 6, 1186 (November, 2003). This degenerative process coincides with a significant reduction of GDNF protein levels in peripheral nerves. It was hypothesized that increasing the levels of GDNF in the skin, where C-fibers terminate, could modify the onset or progression of SFN in these mice. To test this, GFAP-DN-erbB4 mice were crossed with a mouse line that over-expresses GDNF in the skin under the control of the keratin 14 promoter (K14-GDNF) (Zwick et al., J Neurosci 22, 4057 (May 15, 2002). Tests of thermal nociception at six weeks of age showed that GFAP-DN-erbB4 mice exhibited a dramatic loss in thermal nociception), while double transgenic mice (GFAP-DN-erbB4::K14-GDNF) were indistinguishable from wild types and K14-GDNF mice (FIG. 1A). Furthermore, electron microscopy showed that the breakdown of Remak bundles previously documented in GFAP-DN-erbB4 (Chen et al. Nat Neurosci 6, 1186 (November, 2003) was absent in the double transgenic mice (FIG. 1B). Analysis of intra-epidermal nerve fibers (IENF) in glabrous hind paw skin using the neuronal marker protein gene product 9.5 (PGP9.5) showed that GFAP-DN-erbB4 mice have progressive loss of IENFs (FIG. 1E), which was prevented by GDNF overexpression (FIG. 1C and FIG. 1D). Together, the behavioral and structural results demonstrate that GDNF over-expression in the skin prevents both the behavioral and anatomical SFN phenotypes associated with GFAP-DN-erbB4 mice.

### Example 2

### Topical Application of XIB4035 Curtails Progression of SFN in Two Animal Models

The K14-GDNF mice served as a proof-of-concept that GDNF over-expression in the skin could be used to prevent the progressive SFN found in this transgenic line. However, given that K14-GDNF mice overexpress this neurotrophic factor during embryogenesis, it was possible that alterations in sensory neuron development or physiology could have contributed to these results. Since proteins such as GDNF do not readily diffuse through the skin, XIB4035, a reported non-peptidyl small molecule agonist for the GDNF receptor, GFRα1 (Tokugawa et al., Neurochem Int 42, 81 (January, 2003)), was tested as an alternative to GDNF.

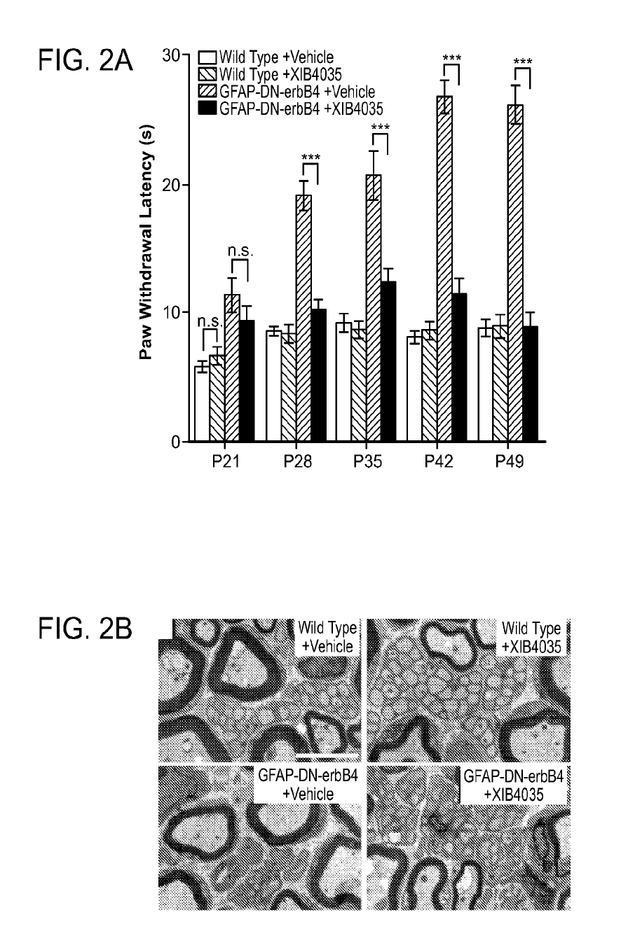

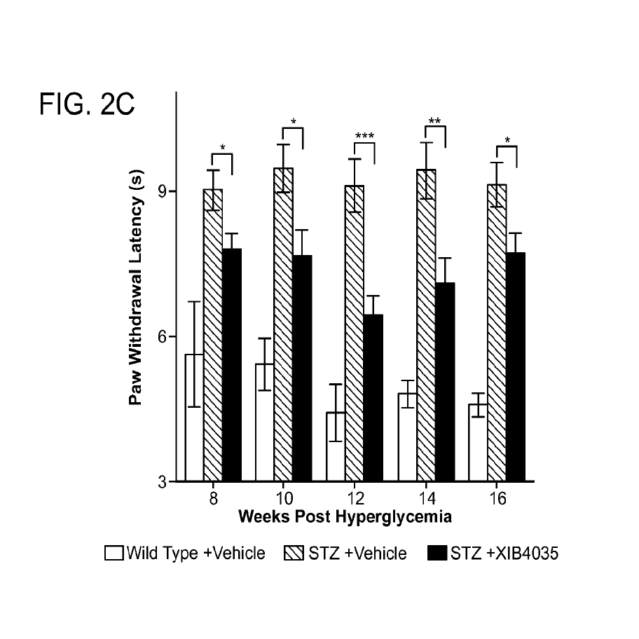

As a first test, a cream containing XIB4035 (1.5 mM) was applied directly to the hind paws of GFAP-DN-erbB4 and wild type mice twice daily for a period of 4 weeks starting prior to symptom onset (P21). Mice of both genotypes were also treated with the base cream containing no drug for control. Mice were tested for responses to a noxious thermal stimulus prior to the initiation of treatment and every 7 days for the duration of the experiment. The behavior of wild type mice remained normal independent of treatment, while GFAP-DN-erbB4 mice treated with control cream progressively lost thermal nociception, as previously reported for untreated animals (Chen, supra) (FIG. 2A). Remarkably, reaction times of GFAP-DN-erbB4 mice treated with XIB4035 remained indistinguishable from wild type animals for the duration of the experiment (FIG. 2A) Importantly, response thresholds to mechanical stimuli were normal in all groups after the treatment period, indicating XIB4035 treatment had no effect on mechano-reception. Furthermore, electron microscopic analysis showed that XIB4035 treatment prevented the degeneration of Remak bundles and C-fiber axons (FIG. 2B). Quantitative EM analysis also showed that XIB4035 treatment preserved both the size of c-fiber axons and the number of c-fibers per Remak bundle in GFAP-DN-erbB4 mice (Table 1).

TABLE 1
Treatment of GFAP-DN-erbB4 mice with XIB4035 preserves
Remak bundle structure in GFAP-DN-erbB4 mice

| | | c-fibers/ |
|---|---|---|
| | c-fiber area¹ | Remak bundle² |
| WT + vehicle | 1.78 ± 0.10 m² | 9.90 ± 0.66 |
| WT + XIB4035 | 1.81 ± 0.06 m² | 8.80 ± 0.52 |
| GFAP-DN-erbB4 + vehicle | 1.01 ± 0.07 m²* | 4.71 ± 0.42* |
| GFAP-DN-erbB4 + XIB4035 | 1.64 ± 0.07 m² | 8.40 ± 0.83 |

Thus, topical treatment with XIB4035 is effective at preventing progressive SFN in GFAP-DN-ErbB4 mice similar to GDNF overexpression in the skin.

To explore the utility of XIB4035 in a more clinically relevant model, diabetic peripheral neuropathy was selected because more than 50% of all diabetic patients develop some form of peripheral neuropathy, particularly SFN. Streptozotocin (STZ) induced diabetic model was chosen, in which a single injection of STZ kills pancreatic beta cells causing diabetes and produces SFN symptoms several weeks later. Upon becoming hyperglycemic, STZ injected mice were topically treated daily with either control or XIB4035 containing cream for 16 weeks. Thermal nociceptive tests began 8 weeks after initiation of treatment and were repeated every two weeks. These tests showed that XIB4035 treated diabetic mice had better sensory function than those exposed to the control cream at the first test, and improvement persisted for the duration of the experiment, indicating that XIB4035 preserved sensory function in diabetic mice in the long-term (FIG. 2C).

### Example 3

### Therapeutic Use of XIB4035 Restores Sensory Behavior in Neuropathic Animals

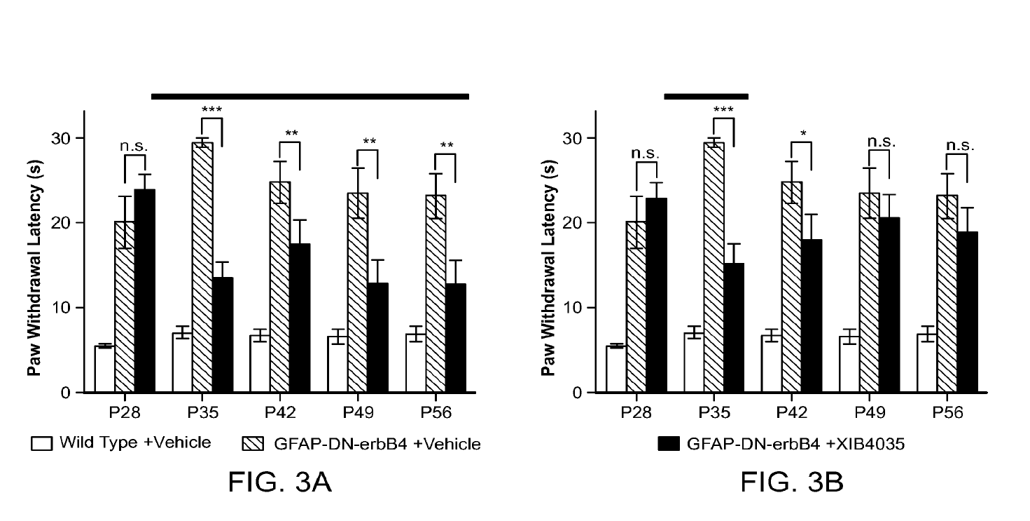

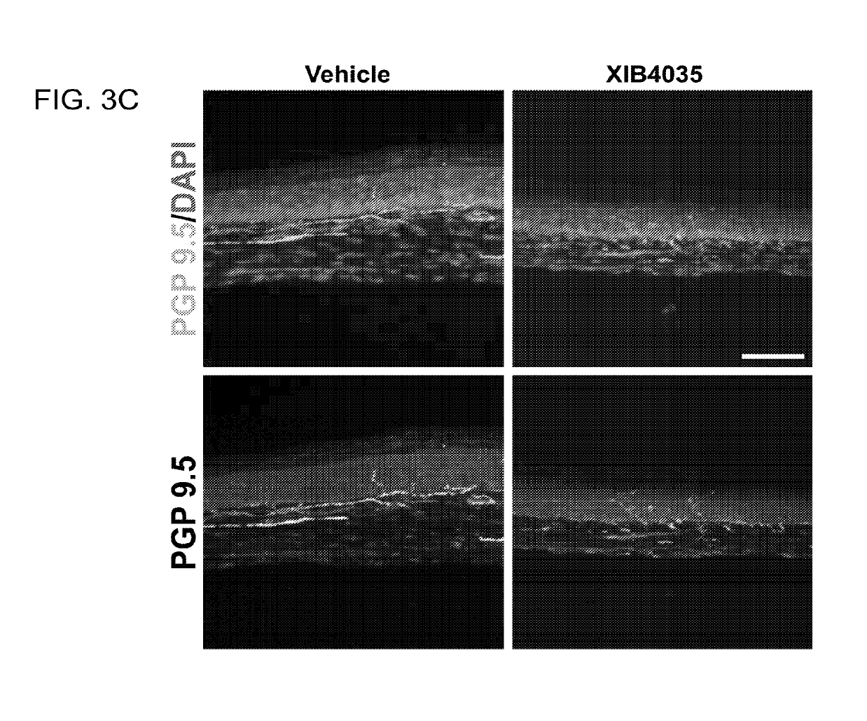

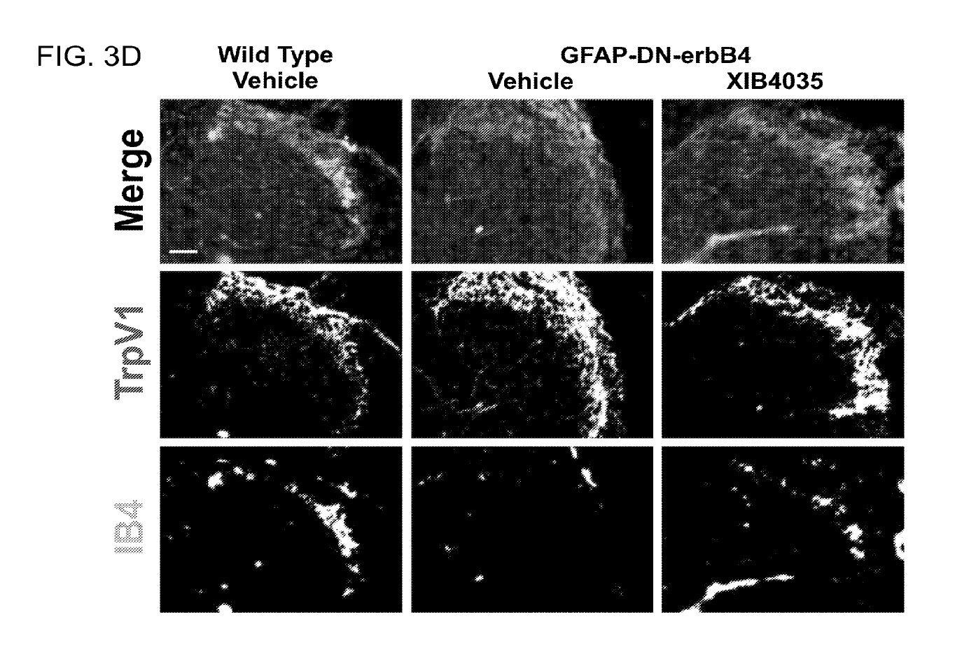

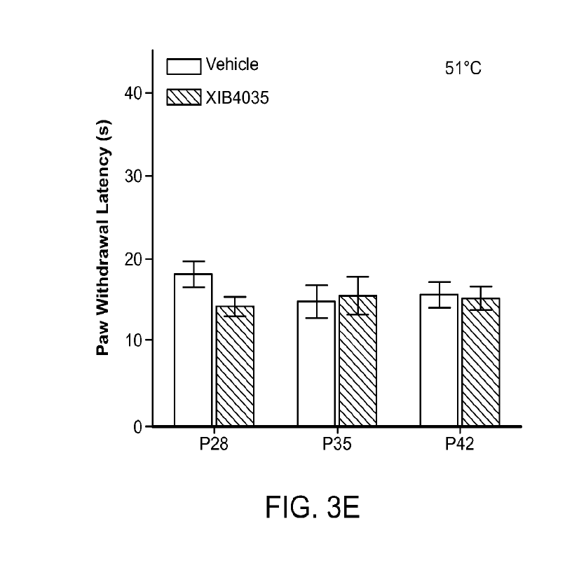

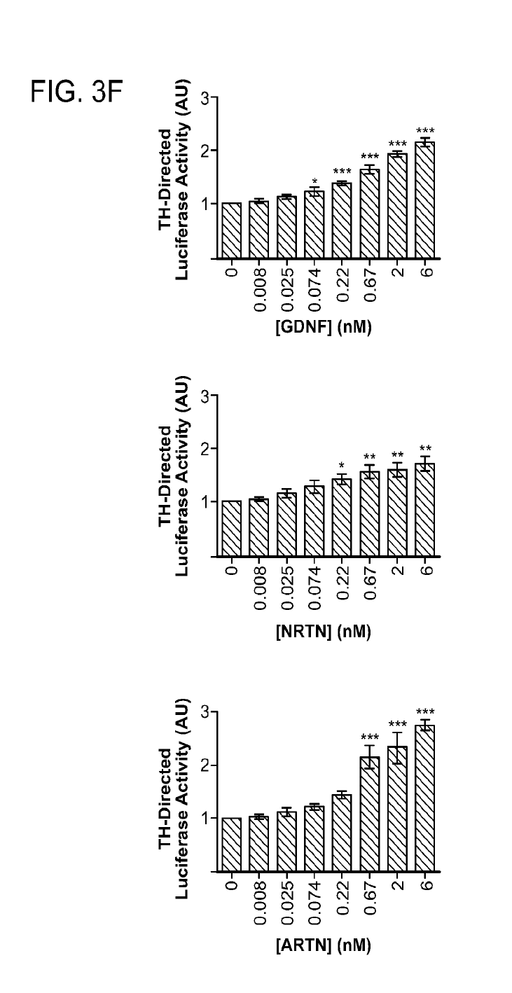

The results described above demonstrated that prophylactic use of topically applied XIB4035 prevents or reduces SFN symptoms in the GFAP-DN-erbB4 and diabetic models. However, in the clinic, therapy would almost certainly begin after patients present with symptoms of SFN. Therefore, it was determined whether topical XIB4035 can act as a disease-modifying agent in the GFAP-DN-erbB4 neuropathy model starting after animals are clearly symptomatic (P28). Remarkably, neuropathic animals showed a significant reduction in symptoms one week after treatment initiation (P35), and the improvement persisted for the duration of the experiment (FIG. 3A). However, if treatment was initiated at P28 but interrupted at P35, neuropathic symptoms reappeared one week later (FIG. 3B), indicating that chronic XIB4035 treatment is necessary to maintain the sensory recovery. Surprisingly, the sensory improvement in this paradigm did not correlate with a recovery of IENFs at P35 (FIG. 3C) or P63. Therefore, to further explore the effects of therapeutic intervention on the structure of sensory neurons, the central projections of C-fibers in the spinal cord were analyzed by staining for isolectin-B4 (IB4), as the majority of GFRα expressing DRG exhibit IB4 binding. While IB4 labeling in mutant mice was lower than wild types regardless of treatment, IB4 signal was clearly present in mutant mice treated with XIB4035 while absent in those treated with control cream (FIG. 3D; 83% of XIB4035 treated mice (n=6) with IB4+ lamina II terminals vs 0% (n=4) in control treated mice) (FIG. 3D). These data demonstrate that XIB4035 is an effective disease-modifying therapy for SFN, and suggest functional recovery is due to improved health and structure of C-fiber central projections.

Importantly, given the potential need for chronic application of XIB4035, the question of whether XIB4035 produces side effects in both mutant and wild type mice was examined. XIB4035 did not induce thermal hyperalgesia in wild types (FIG. 3E), and no changes in appearance, food intake, skin health or other outward negative signs in animals treated twice a day for 7 weeks were detected. Together, these results demonstrate that topical XIB4035 can be used as a disease-modifying agent, both prophylactically and therapeutically, for treating SFN arising from diverse neurological insults, and that the efficacy of treatment is not accompanied by any overt side effects.

### Example 4

### Mechanism of XIB4035 Action: It is not an Agonist for GFRα/RET Receptors

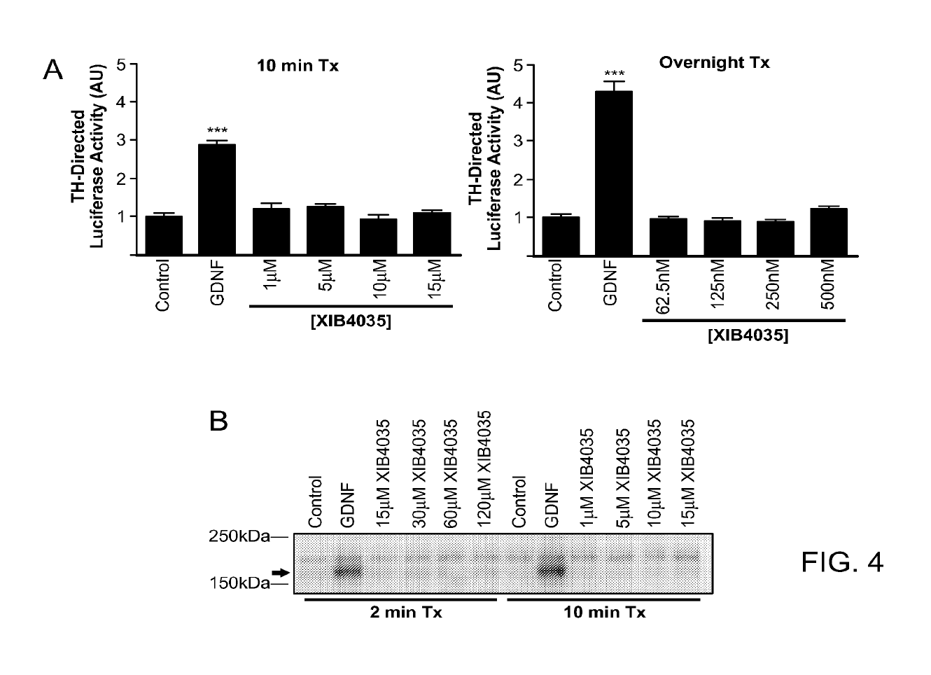

One goal of these studies was to determine if XIB4035 could be used for treating SFN in humans. Using the Neuro2A (N2A) murine neuroblastoma cell line, Tokugawa et al. (supra) reported XIB4035 as a competitive agonist for the GDNF receptor GFRα1 that activates the RET receptor. Since populations of DRG sensory neurons express different GFRα receptors, thus making them preferentially responsive to the particular ligands, it was important to determine if XIB4035 was specific to GFRα1 or could act on other family members. Since PSPN has no effect on peripheral sensory neurons, the GFRα1, GFRα2, and GFRα3 receptors were the focus of these studies. The SH-SY5Y, a human neuroblastoma cell line expressing mRNA for GFRα1, GFRα2, and GFRα3, was used to perform two cell-based assays; immunoblots measuring RET phosphorylation and a luciferase reporter assay using the tyrosine hydroxylase (TH) promoter. For the latter two paradigms were used, either overnight treatment immediately followed by luciferase activity measurements or 10 minute treatments followed by washout and overnight incubation prior to measurements. GDNF and ARTN induced robust TH-luciferase activity in a dose dependent manner (FIG. 3E). Because the responses to GDNF and ARTN were more robust, these two factors were chosen for further analysis. Surprisingly, Contrary to expectations of a GFRα1 agonist, XIB4035 had no effect in the TH-luciferase assay in either the overnight (6.25-500 nM) or 10 minute (1-15 μM) treatment paradigms (FIG. 4A). XIB4035 also failed to induce RET phosphorylation in these cells (FIG. 4B).

### Example 5

### XIB4035 is a Positive Modulator of Ligand Induced GFRα/RET Signaling

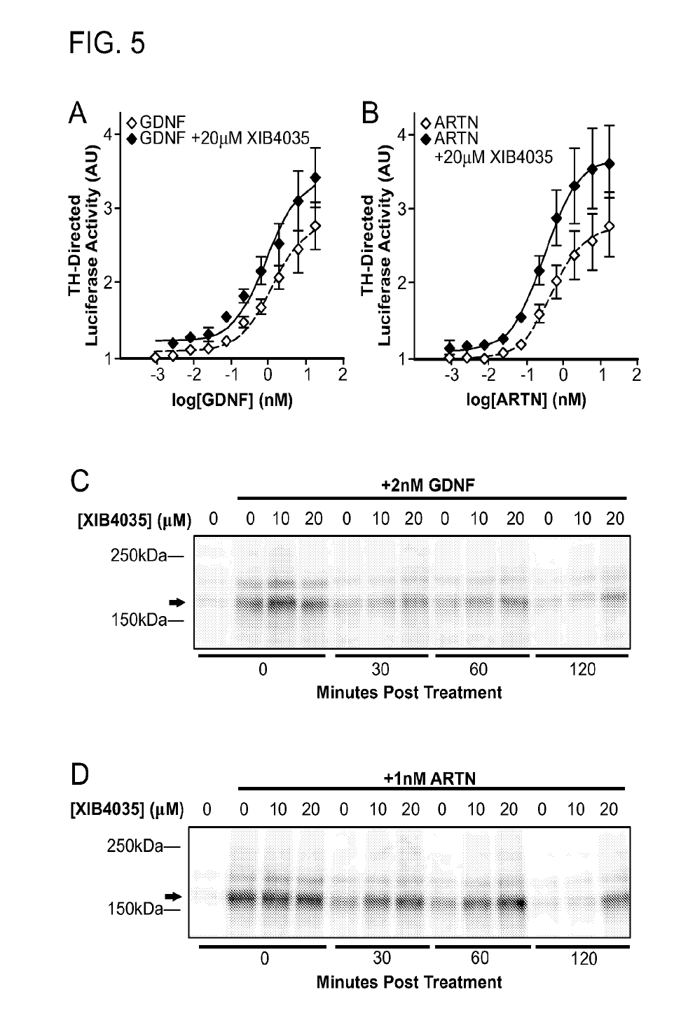

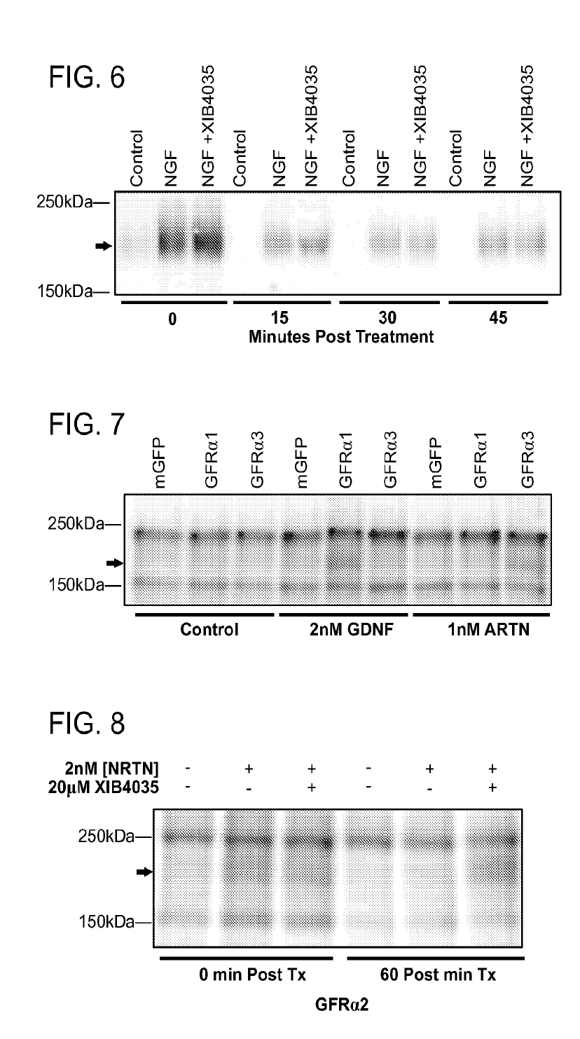

Since the original report argued that XIB4035 displaces GDNF binding to GFRα1 expressing cells (FIG. 2 in Tokugawa et al., Neurochem Int 42, 81-86 (2003).), potentially ARTN, on TH-luciferase activity and Ret phosphorylation. Surprisingly, as reported herein it was found that XIB4035 co-treatment of cells with either GDNF or ARTN significantly potentiates the effects of both ligands on TH-luciferase activity over a range of doses (FIG. 5a and b), resulting in a significant shift in the non-linear regression of the dose-response curve, reduced minimal ligand dose necessary to induce luciferase activity above control, and increased maximal effect. Moreover, Western blot assays revealed that co-treatment with XIB4035 prolongs the GDNF- and ARTN-induced RET phosphorylation (FIGS. 5C and 5D). In this experiment, cells were treated with GDNF or ARTN with or without XIB4035 for 10 minutes and cell lysates were either collected immediately or treatment was washed out and replaced with growth media for 30, 60, or 120 minutes. In cells treated with GDNF or ARTN alone, RET phosphorylation was clearly reduced by 30 minutes and undetectable by 2 hours after ligand removal. In contrast, phosphorylation remained high at 30 minutes and was still detectable at 2 hours in cells co-treated with 20 μM XIB4035 Importantly, XIB4035 did not influence the activity of two other receptor tyrosine kinase pathways, NGF-induced activation of TrkA signaling in PC12 cells and NRG1-dependent activation of ErbB2/ErbB3 in L6 muscle cells (FIG. 6), suggesting that XIB4035 is a specific signaling modulator of GDNF family ligands. The experiments with SH-SY5Y cells indicated that XIB4035 is not a true ligand for either GFRα1, 2, or 3, but that it enhances ligand-induced GFRα/RET signaling. However, the tests on SH-SY5Y cells did not allow us to determine which of the GFRαs is sensitive to XIB4035. Therefore, we tested cells expressing only one GFRα using either N2A cells (which express RET) transfected with GFRα1 or GFRα3 expression constructs, or B(E)2-C cells, which express only GFRα2 together with RET. Initially, we treated control transfected (mGFP) N2A cells with either GDNF or ARTN, and demonstrated that neither ligand induced RET phosphorylation (FIG. 7). In contrast, GDNF and ARTN induced RET phosphorylation when their cognate receptors were expressed (FIG. 7), indicating the lack of functional endogenous GFRαs in these cells. The GFRα1 or GFRα3 transfected N2A cells were then treated with XIB4035 alone, the appropriate ligand for the expressed receptor alone, or XIB4035 and ligand in combination for 10 minutes, and collected lysates immediately after treatment or 60 minutes after washout. XIB4035 alone did not induce RET phosphorylation in either GFRα1 or GFRα3 transfected cells (FIGS. 6A and 6B). However, RET phosphorylation was clearly prolonged at the 60 min time-point when co-treated with XIB4035 and ligand compared to ligand alone, (FIGS. 6A and 6B). Similar results were obtained with GFRα2 and NRTN using B(E)2-C cells (FIG. 8). Thus, XIB4035 is a positive modulator of signaling by GDNF family ligands and their receptors, not an agonist for GFRα1 as previously reported.

The results reported herein provide for a novel therapeutic GFRα/RET signaling modulator XIB4035. Small fiber neuropathy (SFN) is a disorder with complex, multifaceted origins and symptomatic presentation. GDNF family receptors and their co-receptor, RET, are used as therapeutic targets for SFN. These results demonstrate that topical application of GDNF or XIB4035, a non-peptidyl GFRα/RET signaling modulator, attenuated symptomatic pathology in two models of progressive SFN. Furthermore, XIB4035 acts therapeutically in the GFAP-DN-erbB4 mice after onset of SFN symptoms. These results present a novel therapeutic treatment for SFN using topical application of the GFRα/RET signaling modulator, XIB4035. Finally, these results indicate that XIB4035 is not a GFRα1 agonist as previously reported (Tokagawa, supra), but functions to specifically augment ligand stimulated GFRα/RET signaling.

Delivery of neurotrophic factors (GDNF, NGF, and BDNF) has been considered as a strategy for the treatment of a variety of neurological disorders, including neuropathic pain and Parkinson's disease. In particular, GDNF family ligands showed great promise in animal models, but have yet to yield any approved therapies in humans. Two major barriers for moving these therapies to the clinic are target delivery and high systemic doses necessary for efficacy. Previous results demonstrated effectiveness of neurotrophic factor delivery via systemic or intrathecal injection in animal models. Nevertheless, these routes of delivery proved ineffective and/or cause severe side-effects in human patients. For example, trials examining NGF treatment in patients with diabetes-induced peripheral neuropathy showed some improvement in patients' perception of symptom severity, but side-effects including myalgia, peripheral edema, and hyperalgesia were observed. Furthermore, intracerebroventricular administration of GDNF to Parkinson's disease patients resulted in weight loss, anorexia, and nausea with little clinical benefit (Nutt, Neurology 60:69-73, 2003). Systemic delivery of XIB4035 would not be expected to induce these negative side-effects because as an enhancer of endogenous GDNF it is only increasing GDNF activity at sites where GDNF normally binds. The results reported herein indicate that local, directed delivery of molecules that stimulate or enhance GDNF signaling may also address these issues. Furthermore, given that XIB4035 shows remarkable effectiveness in two murine models of SFN with very different pathogenic mechanisms, this drug may be useful in a broad spectrum of SFNs, e.g. those caused by chemotherapy and injury.

The behavioral recovery resulting from XIB4035 treatment following SFN onset is not accompanied by recovery of IENF density, a finding that is consistent with previous reports indicating that absence of IENFs from the skin does not always coincide with hypoalgesia. These results raise a question as to the use of skin biopsies for diagnosing peripheral neuropathies. Nevertheless, XIB4035 treatment produced partial recovery of IB4 positive C-fiber projections in the dorsal horn of the spinal cord, suggesting that this drug positively influences the health and function of nociceptive, GDNF family ligand responsive C-fibers. These data indicate that the overall health of the sensory neurons, particularly their central projections, may be more important than density measurements to development of and recovery from progressive SFN.

The results presented herein regarding the molecular mechanism of XIB4035 action conflict with the only published study pertaining to this molecule. We found that XIB4035 functions as a positive modulator of GDNF family signaling by prolonging ligand-induced RET receptor activation and enhancing downstream effects of this receptor. Determining XIB4035 is not an agonist for GFRα receptors raises the question of how topical treatment with XIB4035 alone leads to the observed therapeutic effects in our SFN models. Without wishing to be bound by theory, it is likely that XIB4035 acts by enhancing signaling of endogenous GDNF family ligands, e.g. GDNF produced by basal keratinocytes.

### Example 6

### Therapeutic Use of XIB4035 Restored Nerve Conduction Velocity (NCV) and Maintained Sub-Epidermal Neural Plexus (SNP) in Diabetic Neuropathic Model Animals

To examine the impact of XIB4035 treatment upon either nerve conduction velocity (NCV) or sub-epidermal neural plexus (SNP) in diabetic neuropathy animal models, the following experiments were performed.

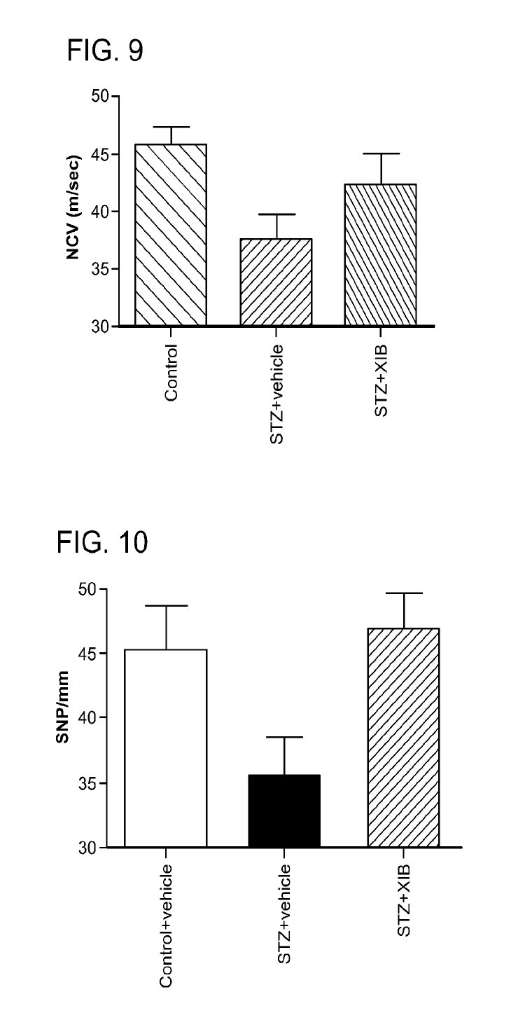

To examine NCV, normo-glycemic or diabetic mice (STZ model, as described elsewhere herein) were treated with either vehicle cream or with cream containing XIB4035 from the moment they became hyperglycemic, for 16 weeks. At the end of this period, nerve conduction velocity (NCV) was measured in sciatic nerves. NCV in diabetic mice without XIB treatment exhibited slower conduction velocity, whereas NCV in diabetic mice administered XIB was not statistically different from that observed in control animals (FIG. 9).

To examine SNP, normo-glycemic or diabetic mice (STZ model) were treated with either vehicle cream of cream containing XIB4035 from the moment they became hyperglycemic, for 16 weeks. At the end of this treatment period, the density of sub-epidermal fibers in papillary dermis was measured in papillary dermis. It was thereby identified that sub-epidermal neural plexus (SNP) density was reduced in diabetic mice not administered XIB, but was preserved in diabetic mice that had received XIB4035 treatment (FIG. 10).

The results reported herein above were obtained using the following materials and methods.

### Animals.

Transgenic mouse lines used were as previously described (Lacomis et al., Muscle Nerve 26, 173-188 (2002); Comblath et al., Current opinion in Neurology 19, 446-450 (2006)). Animals were kept in the animal facility with free access to food and water. Behavioral experiments were performed in a quiet environment at the same time of day. The hot plate test was performed using a “controlled hot-plate analgesia meter” (Columbus Instruments) heated to 54° C. Paw withdrawal latency was measured as the time required for the mouse to visibly respond to the thermal stimulus, e.g. licking paws, shaking paws, or jumping off of the plate.

Mechanical sensitivity was tested by simulation of the plantar surface of the hind paw with a series of von Frey filaments while the animal was placed on an elevated wire grid. The threshold was determined as the lowest force that evoked a visible withdrawal response. The use of animals was approved by the Animal Care and Use Committee of Children's Hospital Boston.

### Diabetic Neuropathy.

Adult female C57 Bl/6J mice were made diabetic (blood glucose >15 mmol/L) by injection of STZ (90 mg/kg i.p.) on two consecutive days, with confirmation of hyperglycemia made 7 days after STZ delivery. Paw thermal response latency of the right paw was measured every two weeks from weeks 8-16 of diabetes using a modified Hargeaves test, as described

### Preparation and Use of XIB4035.

The cream containing XIB4035 (1.5 mM, ZereneX Molecular Ltd., Manchester, UK) consisted of N-methyl-pyrrolidone (6.25%), isopropyl myristate (6.25%) and petroleum jelly (87.5%). Control cream had the same ingredients without XIB4035. Cream was applied twice daily to the hind paws of mice starting at P21 for a period of 4 weeks for prophylactic treatment of GFAP-DN-erbB4. Diabetic mice were treated twice daily for 8 weeks after streptozotocin (STZ) injection prior to onset of hyperglycemia and for another 8 weeks during neuropathy testing. Cream treatment in therapeutic studies using GFAP-DN-erbB4 mice was performed twice daily beginning after onset of SFN at P28 and either continued for 5 weeks (chronic treatment) or 1 week (acute treatment).

### Plastic Embedding and Electron Microscopy.

Tissue was prepared as in (Lacomis, supra). Briefly, mice were perfused intracardially with 2% paraformaldehyde, 2.5% gluteraldehyde and 0.03% picric acid in 0.1 M cacodylate buffer (pH 7.2). Tissue was post-fixed overnight at 4° C. and embedded in Epon. Ultrathin sections were cut, collected on cellodin-coated grids and stained using uranyl acetate and lead citrate. Photographs were taken using the Tecnai G2 Spirit BioTWIN transmission electron microscope.

### Immunohistochemistry.

Mice were anesthetized with 2.5% Avertin. Hind paws were removed, immersion fixed in 2% paraformaldehyde, 14% picric acid in 0.1 M phosphate buffer (pH 7.4) overnight at 4° C., and cryoprotected in 20% sucrose overnight at 4° C. Footpad skin was dissected from hind paws, embedded in OCT, sectioned at 30 μm, and stained as floating sections. Tissue was blocked for 30 minutes in 0.1M PB+0.3% Triton-X 100+10% normal goat serum and incubated with PGP9.5 rabbit polyclonal antibody (Ultraclone, 1:1000), overnight at 4° C. Sections were washed 3 times for 10 minutes in 0.1M PB+0.3% Triton-X 100+10% normal goat serum followed by incubation with goat anti-rabbit Alexa-488 (Invitrogen) 1:1000 for 1 hour at room temperature.

Spinal cords were collected from mice anesthetized with 2.5% Avertin, immersion fixed in 4% paraformaldehyde in 0.1 M phosphate buffer (pH 7.4) for 2 hours at 4° C., and cryoprotected in 20% sucrose overnight at 4° C. Tissue was embedded in OCT, sectioned at 15 μm, and stained mounted on slides. Tissue was blocked for 30 minutes in 0.1M PB+0.3% Triton-X 100+10% normal goat serum and incubated with TrpV1 rabbit polyclonal antibody (AbCAM, 1:2000) and Alexa-488 conjugated isolectin-B4 overnight at 4° C. Sections were washed 3 times for 30 minutes in 0.1M PB+0.3% Triton-X 100+10% normal goat serum followed by incubation with goat anti-rabbit Alexa-594 (Invitrogen) 1:1000 for 1 hour at room temperature.

In all experiments, nuclei were stained with DAPI during secondary antibody incubations and sections were mounted with glycerol-based mounting media containing 1% (w/v) phenylenediamine.

### Immunohistochemistry Analysis.

Skin section images were acquired as 30 μm Z-stacks (1 μm intervals) and processed as maximum intensity projections using a Zeiss LSM 700 microscope and ZEN software. Acquisition measures were set using control treated wild-type sections and used for all sections imaged. A measured line was drawn using the DAPI channel to delineate the border between basal keratinocytes and outer layers of the epidermis. All PGP9.5 positive IENFs crossing the border were counted and expressed as a number per 100 μm length. Analysis of IENF density was blinded to genotype and treatment.

Spinal cord images were acquired using a Zeiss Axioscope microscope. Exposure times for each channel were set using wild-type control treated sections and used for all images. Analysis of the presence of dorsal horn IB4 staining was blinded to genotype and treatment.

### Promoter Cloning and Stable Cell Generation.

The tyrosine hydroxylase promoter was cloned into the pGL3 basic vector (Promega, Madison, Wis., USA) at the Mlu I and Hind III sites, as previously described (Zwick, supra) (TH-pGL3). Briefly, a 2 Kb promoter region upstream from the transcription initiation site of the rat TH gene was cloned from genomic DNA using the primer sequences, TGACGCGTAGGCACAGCTCCCTCCTACCCCGT and AGAAGCTTCCCTCGCCAGGCAGGCGCCCTCT. SH-SY5Y cells were co-transfected with the TH-pGL3 and the pBabe-puromycin expression vectors at a molar ratio of 10:1. Cells were selected for stable puromycin resistance at a final concentration of 0.5 μg/ml of puromycin-dihydrochloride. Stable colonies were selected and tested for TH-directed luciferase response to GDNF family ligand stimulation. One stable clone, SH-SY5Y-THpGL3, with good signal-to-noise ratio was selected for use in the experiments.

### Cell Assays.

SH-SY5Y-THpGL3 cells were maintained in DMEM/F12, 5% FBS, and 1% penicillin/streptomycin and plated on collagen coated plates (4 μg/ml). Neuro-2A cells were grown in MEM, 5% FBS, and 1% penicillin/streptomycin. BE(2)-C cells were grown in DMEM/F12 media containing 5% FBS and 1% penicillin/streptomycin. PC-12 cells were maintained in media containing RPMI-1640, 10% horse serum, 5% FBS, and 1% penicillin/streptomycin on collagen coated plates. For luciferase assays, cells were treated for either 10 minutes with washout or overnight with various molecules and assayed for firefly luciferase 16-24 hours post-treatment using the luciferase assay system (Promega, Madison, Wis., USA).

For phosphorylated Ret immunoblot experiments, cells were treated with various combinations of molecules for 10 minutes and either collected immediately or had treatment washed out and returned to control treatment media for the times indicated prior to cell collection. Lysates were collected in buffer containing: 50 mM Tris-HCL, 1% TX-100, 0.25% Deoxycholic acid, 150 mM sodium chloride, 1 mM EDTA, 0.1 mM sodium fluoride, 0.1 mM sodium pyrophosphate, 0.02 mM sodium orthovanadate, and protease inhibitors Immunoblots were blocked in 5% bovine serum albumin in tris-buffered saline+0.2% Tween 20+0.1 mM sodium fluoride, 0.1 mM sodium pyrophosphate, and 0.02 mM sodium orthovanadate and probed in blocking solution using anti-phospotyrosine (4G10) mouse monoclonal antibody (1:2000; EMD Milipore Corp., Billerica, Mass.). Secondary detection was performed in blocking solution using HRP-conjugated goat-anti-mouse antibodies (1:1000; MP Biolomedicals LLC, Solon, Ohio).

N2A cells were transfected with expression plasmids expressing a membrane bound form of EGFP (mGFP), rat GFRα1, or human GFRα3 (GFR constructs were kind gifts from Dr. Jefferey Milbrandt at Washington University School of Medicine, St Louis, Mo.). N2A and PC-12 cells were starved in basal media containing 1% FBS overnight prior to treatments. SH-SY5Y-THpGL3 and BE(2)-C cells were treated in growth media. N2A and PC12 cells were treated in starvation media. GDNF, NRTN and ARTN were purchased from Peprotech. 50 μg/ml of β-NGF (R&D Systems, Inc.) was used to stimulate TrkA receptor phosphorylation in PC-12 cells.

### Statistical Analyses.

All statistical analyses were performed using Prism 4 (GraphPad Software, Inc.). ANOVA post-hoc tests are indicated. SEM was used to indicate error in all analyses as n≧3. P-values for Student's t-tests are indicated as actual values in text and figure legends. For FIG. 5a and b, comparison of the non-linear curve regression was performed using an FTest. Analyses of the minimal ligand dose necessary to induce significant luciferase activity (FIG. 5 a and b) was performed by Student's t-test comparing the average fold luciferase induction from three experiments of individual treatments to control (non-treated).

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for treating a neuropathy, the method comprising administering an effective amount of a combination of XIB4035 and a GFRa ligand to a subject in need thereof, thereby treating the neuropathy.

2. (canceled)

3. The method of claim 1, wherein XIB4035 enhances the activity of a GFRa ligand selected from the group consisting of GDNF, neublastin, neuturin (NRTN), artemin (ARTN), and persephin.

4. The method of claim 1, wherein XIB4035 enhances the activity of ligand-induced GFRa/Ret signaling.

5. The method of claim 4, wherein the GFRa is GFRa1, GFRa2, GFRa3, or GFRa4.

6. The method of claim 1, wherein the subject is identified as having a neuropathy selected from the group consisting of diabetic small fiber neuropathy, injury-associated neuropathy, alcoholism-associated neuropathy, lupus-related neuropathy, HIV-related neuropathy, large fiber neuropathy, a neuropathy associated with chemotherapy and enteric neuropathy.

7. The method of claim 1, wherein an effective amount of XIB4035 is between about 0.5 and 3 μM.

8. The method of claim 1, wherein an effective amount results in a plasma concentration of 6.92-16.93 ng/ml at 6-12 hours after dosage.

9. The method of claim 1, wherein an effective amount of XIB4035 is 1.5 μM.

10. The method of claim 1, wherein the amount of XIB4035 is sufficient to relieve symptoms of neuropathy.

11. The method of claim 1, wherein XIB4035 is administered systemically.

12. The method of claim 1, wherein XIB4035 is administered orally, intravenously, intramuscular, subdermally, or intrathecally.

13. The method of claim 1, wherein XIB4035 is administered once per day.

14. The method of claim 1, wherein the method further comprises administering GDNF.

15. The method of claim 14, wherein GDNF is administered locally.

16. The method of claim 14, wherein the GDNF polypeptide is administered by injection into a ventricle of the brain, into cerebrospinal fluid, or locally using an implanted pump or matrix.

17. The method of claim 1, wherein GDNF is administered using an expression vector comprising a polynucleotide encoding GDNF.

18. The method of claim 17, wherein the expression vector comprises a promoter that directs expression in muscle or skin.

19. The method of claim 10, wherein the subject is identified as having a large fiber or other neuropathy by electrodiagnostic testing, sensory, motor nerve conduction, F response, H reflex, needle electromyography (EMG), and/or clinical indications.

20-21. (canceled)

22. A composition for the treatment of neuropathy, the composition comprising an effective amount of XIB4035 and GDNF.

23. A kit for the treatment of neuropathy, the kit comprising an effective amount of XIB4035 and GDNF.

24. A method selected from the group consisting of:
a method for treating a large fiber neuropathy, the method comprising administering to a subject identified as in need thereof an effective amount of XIB4035 or a compound of Tables 1-3, thereby treating the large fiber neuropathy;
a method for improving nerve conduction velocity (NCV) in a subject having a neuropathy, comprising administering to said subject an amount of XIB4035 sufficient to improve nerve conduction velocity in said subject;
a method for increasing sub-epidermal neural plexus (SNP) density in a subject having or at risk of having a neuropathy, comprising administering to said subject an amount of XIB4035 sufficient to increase SNP density in said subject; and
a method for maintaining sub-epidermal neural plexus (SNP) density in a subject having or at risk of having a neuropathy comprising administering to said subject an amount of XIB4035 sufficient to maintain SNP density in said subject.

25. The method of claim 24, wherein nerve conduction velocity is improved in the sciatic nerve of said subject.

26. The method of claim 24, wherein said subject has a diabetic neuropathy.

27. The method of claim 24, wherein said NCV is at least 40 m/sec, at least 41 m/sec, at least 42 m/sec, at least 43 m/sec, at least 44 m/sec or at least 45 m/sec.

28-29. (canceled)

30. The method of claim 24, wherein said subject has a diabetic neuropathy.

31. The method of claim 24, wherein said SNP density is at least 40/mm, at least 41/mm, at least 42/mm, at least 43/mm, at least 44/mm or at least 45/mm.
